(19) United States
(12) Patent Application Publication
(10) Pub. No.: US 2011/0002995 A1
(43) Pub. Date: Jan. 6, 2011

(54) PHARMACEUTICAL COMPOSITION FOR THE TREATMENT AND PREVENTION OF CARDIAC DISEASE

(75) Inventors: Taehwan Kwak, Gyeonggi-do (KR); Myung-Gyu Park, Gyeonggi-do (KR)

Correspondence Address: LADAS & PARRY, 5670 WILSHIRE BOULEVARD, SUITE 2100, LOS ANGELES, CA, 90036-5679, US

(73) Assignees: MAZENCE INC., DAEJEON (KR); KT & G CORPORATION, Daejeon (KR)

(21) Appl. No.: 12/747,874

(22) PCT Filed: Dec. 18, 2008
(86) PCT No.: PCT/KR2008/007506

§ 371 Date: Sep. 20, 2010

(30) Foreign Application Priority Data
Dec. 31, 2007 (KR) 10-2007-0141303

(51) Int. Cl. A61K 9/14; A61P 9/04; A61P 9/10; A61K 31/352; C07D 311/92; A61K 31/343; C07D 307/92; C07D 311/78; C07D 311/96; C07D 335/08; A61K 31/382; C07D 333/74; A61K 31/381; C07D 327/06; A61K 31/39; C07D 405/12; A61K 31/4178

(52) U.S. Cl. 424/484; 424/489; 514/454; 549/389; 514/468; 549/458; 514/453; 549/384; 549/331; 549/26; 514/437; 549/44; 514/443; 549/16; 514/434; 548/311.4; 514/397; 977/773

(57) ABSTRACT

Provided is a pharmaceutical composition for the treatment and prevention of cardiac diseases, containing (a) a therapeutically effective amount of a compound represented by Formula 1 or 2 or a pharmaceutically acceptable salt, prodrug, solvate or isomer thereof, and (b) a pharmaceutically acceptable carrier, diluent or excipient or any combination thereof.

### FIELD OF THE INVENTION

The present invention relates to a pharmaceutical composition for the treatment and prevention of cardiac diseases. More specifically, the present invention relates to a pharmaceutical composition having excellent effects for the treatment and prevention of cardiac diseases, containing (a) a therapeutically effective amount of a naphthoquinone-based compound or a pharmaceutically acceptable salt, prodrug, solvate or isomer thereof as an active ingredient, and (b) a pharmaceutically acceptable carrier, diluent or excipient or any combination thereof.

### BACKGROUND OF THE INVENTION

Heart is an important organ responsible for systemic blood circulation by receiving blood from veins and continuously supplying the blood to the entire body through arteries. The blood pumped by the heart carries oxygen and various nutritive substances from one part of the body to another and simultaneously carries waste products from various organs or tissues of the body and discharges them to the outside of the body via the kidney or lung.

The wall of the heart is composed of three layers; epicardium (external), myocardium (middle) and endocardium (inner). The endocardium is partially provided with folds and has valves responsible for the opening and closing of the heart.

Pumping of blood into the circulatory system is carried out with cardiac contraction that is caused by the myocardium corresponding to the middle muscular layer of the heart wall. The myocardium is the thickest layer of the heart.

When ventricular load is increased due to hypertension or valvular heart diseases, or dysfunction of cardiomyocytes per se occurs due to myocardial infarction, myocarditis or cardiomyopathy, sufficient amounts of blood cannot be supplied to systemic organs of the body, resulting in reduction of the cardiac output. Subsequently, the body responds to maintain adequate ventricular output, in a manner of heart hypertrophy that results from hypertrophy of cardiomyocytes. The heart is a fully differentiated organ in terms of embryology and therefore cannot further undergo cell proliferation. For these reasons, when there is a need to enhance the cardiac function (cardiac output), the only solution is to increase sizes of existing cardiomyocytes to thereby enhance the myocardial contractility, and such a physiological phenomenon observed in body is called “myocardial hypertrophy”.

Long-term duration of the myocardial hypertrophy is likely to result in high risk of heart failure. Heart failure refers to a condition which is clinically manifested with thinning of the heart wall due to cell apoptosis, and enlargement of atrial and ventricular cavities, thus resulting in significant deterioration of cardiac function. That is, it is known that if no relevant treatments are made to correct myocardial hypertrophy, ventricular hypertrophy may become maladaptive and therefore contribute to the incidence of heart failure resulting from continued ventricular systolic and diastolic dysfunction. Further, due to increases of the ventricular stiffness at the stage of ventricular hypertrophy, heart failure may also be caused by cardiac diastolic dysfunction and failure.

Materials increasing the myocardial contractility or inducing reduction of the cardiac load have been conventionally used for the treatment of cardiac diseases such as myocardial hypertrophy, heart failure, etc. Representative examples of these materials may include digitalis glycosides such as Digoxin and Digitoxin, and PDE3 inhibitors such as Amrinone and Milrinone.

The digitalis glycosides inhibit the Na,K-ATPase to thereby increase an intracellular concentration of Ca²⁺ in cardiomyocytes, which enhances the myocardial contractility, thus treating cardiac diseases. The PDE3 inhibitors increase an intracellular concentration of cAMP to enhance the myocardial contractility and simultaneously they relax vascular smooth muscles (VSMCs) to lower right and left ventricular pressure, which can lead to decreases of the cardiac load and increases of the cardiac output.

In addition, other drugs have been used for the treatment of cardiac diseases, such as beta-adrenalin receptor agonists (e.g. dobutamine), beta-adrenalin receptor blockers, vasodilators, renin-angiotensin inhibitors, and diuretics.

Since a great number of factors are involved in the pathogenic mechanism of heart hypertrophy in living organisms, antagonism against a single pathogenic factor is not sufficient to manage the disease of interest. In addition, although cardiac contractility-improving substances may exhibit quick symptom-relieving effects, prevalence rate and mortality of patients by cardiac diseases such as heart failure are still very high, with very high risk of sudden death within from several months to several years.

Further, patients with end-stage heart failure are in a state with an extreme decline of cardiac function, so heart transplantation is the only treatment scheme. Unfortunately, the number of donor hearts available is extremely limited, so there is no alternative option but to use artificial hearts. Further, post-surgery results are not always satisfactory.

Upon considering that conventional treatments do not provide sufficiently satisfactory results from the viewpoint of achieving long-term purposes and goals for complete recovery of concerned diseases and life-extending, there is an urgent need for development of active agents for the treatment of cardiac diseases such as heart hypertrophy and heart failure.

To this end, the inventors of the present invention have discovered that certain naphthoquinone compounds can exhibit excellent prophylactic and therapeutic effects against cardiac diseases related to heart hypertrophy and heart failure.

Meanwhile, some of pharmaceutical compositions containing conventional naphthoquinone-based compounds as an active ingredient are known in the art. Of these naphthoquinone-based compounds, (β-lapachone is derived from the laphacho tree (Tabebuia avellanedae) which is native to South America, and dunnione and α-dunnione are also derived from the leaves of Streptocarpus dunnii native to South America. These naturally-occurring tricyclic naphthoquinone derivatives have been used for a long time, not only as anti-cancer medications, but also as medications for the treatment of a Chagas disease known as a representative endemic disease of South America, and were also known to exhibit potent efficacies. In particular, pharmacological actions of these naphthoquinone derivatives as anticancer medications have drawn a great deal of attention since they were known to Western nations. As disclosed in U.S. Pat. No. 5,969,163, a number of anti-cancer drugs employing the tricyclic naphthoquinone derivatives are being actually developed by many research groups.

Despite the various researches carried out in the related area, there is no report demonstrating that these naphthoquinone-based compounds exhibit pharmacologically beneficial effects on the treatment or prevention of cardiac diseases associated with heart hypertrophy and heart failure.

### SUMMARY OF THE INVENTION

As a result of a variety of extensive and intensive studies and experiments to solve the problems as described above, the inventors of the present invention have newly demonstrated that certain naphthoquinone-based compounds can be used for the treatment or prevention of cardiac diseases, and have discovered that these compounds can exert desired pharmacological effects, when formulated to be absorbable into target sites of the body. The present invention has been completed based on these findings.

In accordance with an aspect of the present invention, the above and other objects can be accomplished by the provision of a pharmaceutical composition for the treatment and prevention of cardiac diseases, comprising: (a) a therapeutically effective amount of one or more selected from compounds represented by Formulae 1 and 2 below: or a pharmaceutically acceptable salt, prodrug, solvate or isomer thereof; and

(b) a pharmaceutically acceptable carrier, diluent or excipient or any combination thereof.

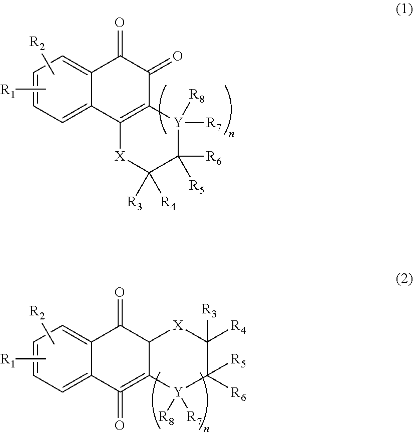

wherein:

R₁ and R₂ are each independently hydrogen, halogen, hydroxyl, or C₁-C₆ lower alkyl or alkoxy, or R₁ and R₂ may be taken together to form a substituted or unsubstituted cyclic structure which may be saturated or partially or completely unsaturated;

R₃, R₄, R₅, R₆, R₇ and R₈ are each independently hydrogen, hydroxyl, C₁-C₂₀ alkyl, alkene or alkoxy, or C₄-C₂₀ cycloalkyl, heterocycloalkyl, aryl or heteroaryl, or two of R₃ to R₈ may be taken together to form a cyclic structure which may be saturated or partially or completely unsaturated;

X is selected from the group consisting of C(R)(R′), N(R″) wherein R, R′ and R″ are each independently hydrogen or C₁-C₆ lower alkyl, O and S, preferably O or S, more preferably O;

Y is C, S or N, with the proviso that R₇ and R₈ are absent when Y is S, and R₇ is hydrogen or C₁-C₆ lower alkyl and R₈ is absent when Y is N; and

n is 0 or 1, with the proviso that when n is 0, carbon atoms adjacent to n form a cyclic structure via a direct bond.

From the experiments conducted to investigate therapeutic effects of a pharmaceutical composition in accordance with the present invention on cardiac diseases, the inventors of the present invention have discovered that administration of the pharmaceutical composition of the present invention significantly decreases the heart weight and size and increases the cardiac contractility in cardiac disease-induced animal experiments, thereby confirming beneficial therapeutic effects on cardiac diseases such as heart hypertrophy and heart failure.

Accordingly, the pharmaceutical composition in accordance with the present invention can be therapeutically or prophylactically used for various kinds of cardiac diseases. In the context of the present invention, the term “cardiac disease” is a broad concept encompassing all kinds of cardiac diseases and disorders and may include, for example, heart hypertrophy, heart failure, congestive heart failure, angina pectoris, myocardial infarction, etc. Preferred is heart hypertrophy or heart failure.

As used the present disclosure, the term “pharmaceutically acceptable salt” means a formulation of a compound that does not cause significant irritation to an organism to which it is administered and does not abrogate the biological activity and properties of the compound. Examples of the pharmaceutical salt may include acid addition salts of the compound with acids capable of forming a non-toxic acid addition salt containing pharmaceutically acceptable anions, for example, inorganic acids such as hydrochloric acid, sulfuric acid, nitric acid, phosphoric acid, hydrobromic acid and hydroiodic acid; organic carbonic acids such as tartaric acid, formic acid, citric acid, acetic acid, trichloroacetic acid, trifluoroacetic acid, gluconic acid, benzoic acid, lactic acid, fumaric acid, maleic acid and salicylic acid; or sulfonic acids such as methanesulfonic acid, ethanesulfonic acid, benzenesulfonic acid and p-toluenesulfonic acid. Specifically, examples of pharmaceutically acceptable carboxylic acid salts include salts with alkali metals or alkaline earth metals such as lithium, sodium, potassium, calcium and magnesium, salts with amino acids such as arginine, lysine and guanidine, salts with organic bases such as dicyclohexylamine, N-methyl-D-glucamine, tris(hydroxymethyl)methylamine, diethanolamine, choline and triethylamine. The compounds in accordance with the present invention may be converted into salts thereof, by conventional methods well-known in the art.

As used herein, the term “prodrug” means an agent that is converted into the parent drug in vivo. Prodrugs are often useful because, in some situations, they may be easier to administer than the parent drug. They may, for instance, be bioavailable by oral administration, whereas the parent may be not. The prodrugs may also have improved solubility in pharmaceutical compositions over the parent drug. An example of a prodrug, without limitation, would be a compound of the present invention which is administered as an ester (the “prodrug”) to facilitate transport across a cell membrane where water-solubility is detrimental to mobility, but which then is metabolically hydrolyzed to the carboxylic acid, the active entity, once inside the cell where water solubility is beneficial. A further example of the prodrug might be a short peptide (polyamino acid) bonded to an acidic group, where the peptide is metabolized to reveal the active moiety.

As an example of such prodrug, the pharmaceutical compounds in accordance with the present invention can include a prodrug represented by Formula 1a below as an active material:

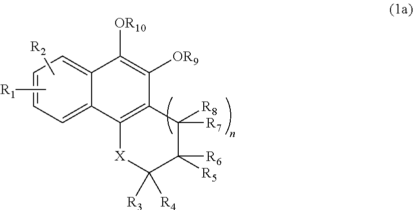

wherein,

R₁, R₂, R₃, R₄, R₅, R₆, R₇, R₈, X and n are as defined in Formula 1.

R₉ and R₁₀ are each independently —SO₃⁻Na⁺, or a substituent represented by Formula A or a salt thereof,

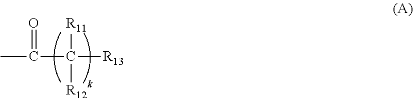

wherein,

R₁₁ and R₁₂ are each independently hydrogen or substituted or unsubstituted C₁-C₂₀ linear alkyl or C₁-C₂₀ branched alkyl,

R₁₃ is selected from the group consisting of substituents i) to viii) below:
i) hydrogen;
ii) substituted or unsubstituted C₁-C₂₀ linear alkyl or C₁-C₂₀ branched alkyl;
iii) substituted or unsubstituted amine;
iv) substituted or unsubstituted C₃-C₁₀ cycloalkyl or C₃-C₁₀ heterocycloalkyl;
v) substituted or unsubstituted C₄-C₁₀ aryl or C₄-C₁₀ heteroaryl;
vi) —(CRR′—NR″CO)₁—R₁₄, wherein R, R′ and R″ are each independently hydrogen or substituted or unsubstituted C₁-C₂₀ linear alkyl or C₁-C₂₀ branched alkyl, R₁₄ is selected from the group consisting of hydrogen, substituted or unsubstituted amine, cycloalkyl, heterocycloalkyl, aryl and heteroaryl, 1 is selected from the 1˜5;
vii) substituted or unsubstituted carboxyl;
viii) —OSO₃—Na⁺;

k is selected from the 0˜20, with proviso that when k is 0, R₁₁ and R₁₂ are not anything, and R₁₃ is directly bond to a carbonyl group.

As used herein, the term “solvate” means a compound of the present invention or a salt thereof, which further includes a stoichiometric or non-stoichiometric amount of a solvent bound thereto by non-covalent intermolecular forces. Preferred solvents are volatile, non-toxic, and/or acceptable for administration to humans. Where the solvent is water, the solvate refers to a hydrate.

As used herein, the term “isomer” means a compound of the present invention or a salt thereof that has the same chemical formula or molecular formula but is optically or sterically different therefrom. Unless otherwise specified, the term “compound of Formula 1 or 2” is intended to encompass a compound per se, and a pharmaceutically acceptable salt, prodrug, solvate and isomer thereof.

As used herein, the term “alkyl” refers to an aliphatic hydrocarbon group. The alkyl moiety may be a “saturated alkyl” group, which means that it does not contain any alkene or alkyne moieties. Alternatively, the alkyl moiety may also be an “unsaturated alkyl” moiety, which means that it contains at least one alkene or alkyne moiety. The term “alkene” moiety refers to a group in which at least two carbon atoms form at least one carbon-carbon double bond, and an “alkyne” moiety refers to a group in which at least two carbon atoms form at least one carbon-carbon triple bond. The alkyl moiety, regardless of whether it is substituted or unsubstituted, may be branched, linear or cyclic.

As used herein, the term “heterocycloalkyl” means a carbocyclic group in which one or more ring carbon atoms are substituted with oxygen, nitrogen or sulfur and which includes, for example, but is not limited to furan, thiophene, pyrrole, pyrroline, pyrrolidine, oxazole, thiazole, imidazole, imidazoline, imidazolidine, pyrazole, pyrazoline, pyrazolidine, isothiazole, triazole, thiadiazole, pyran, pyridine, piperidine, morpholine, thiomorpholine, pyridazine, pyrimidine, pyrazine, piperazine and triazine.

As used herein, the term “aryl” refers to an aromatic substituent group which has at least one ring having a conjugated pi (π) electron system and includes both carbocyclic aryl (for example, phenyl) and heterocyclic aryl(for example, pyridine) groups. This term includes monocyclic or fused-ring polycyclic (i.e., rings which share adjacent pairs of carbon atoms) groups.

As used herein, the term “heteroaryl” refers to an aromatic group that contains at least one heterocyclic ring.

Examples of aryl or heteroaryl include, but are not limited to, phenyl, furan, pyran, pyridyl, pyrimidyl and triazyl.

R₁, R₂, R₃, R₄, R₅, R₆, R₇ and R₈ in Formula 1 or 2 in accordance with the present invention may be optionally substituted. When substituted, the substituent group(s) is (are) one or more group(s) individually and independently selected from cycloalkyl, aryl, heteroaryl, heteroalicyclic, hydroxy, alkoxy, aryloxy, mercapto, alkylthio, arylthio, cyano, halogen, carbonyl, thiocarbonyl, O-carbamyl, N carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino including mono and di substituted amino, and protected derivatives thereof. Further, substituents of R₁₁, R₁₂ and R₁₃ in the Formula 1a may be also substituted as defined in above, and when substituted, they can be substituted as the substituents mentioned above.

Among compounds of Formula 1, preferred are compounds of Formulas 3 and 4 below.

Compounds of Formula 3 are compounds wherein n is 0 and adjacent carbon atoms form a cyclic structure (furan ring) via a direct bond therebetween and are often referred to as “furan compounds” or “furano-o-naphthoquinone derivatives” hereinafter.

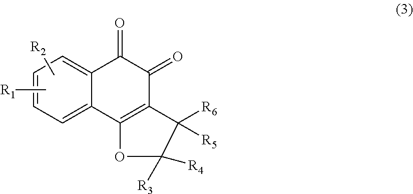

Compounds of Formula 4 are compounds wherein n is 1 and are often referred to as “pyran compounds” or “pyrano-o-naphthoquinone” hereinafter.

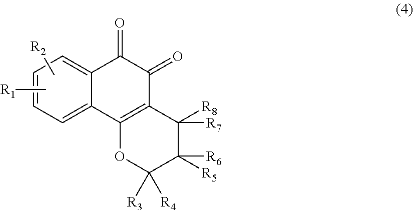

In Formula 1, each of R₁ and R₂ is particularly preferably hydrogen.

Among the furan compounds of Formula 3, particularly preferred are compounds of Formula 3a wherein R₁, R₂ and R₄ are hydrogen, or compounds of Formula 3b wherein

R₁, R₂ and R₆ are hydrogen.

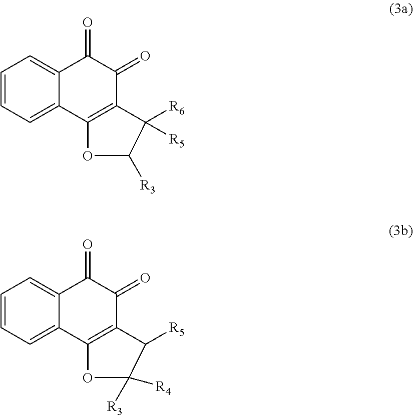

Further, among the pyran compounds of Formula 4, particularly preferred is compounds of Formula 4a wherein R₁, R₂, R₅, R₆, R₇ and R₈ are hydrogen.

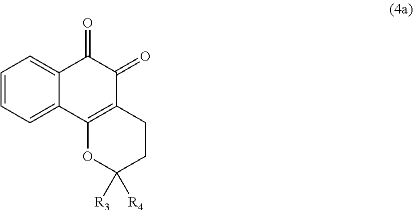

Among compounds of Formula 2, preferred without limitation, are compounds of Formulas 2a and 2b below.

Compounds of Formula 2a are compounds wherein n is 0 and adjacent carbon atoms form a cyclic structure via a direct bond therebetween and Y is C.

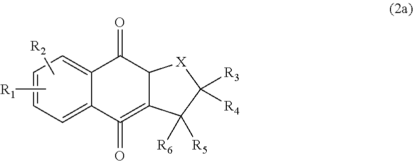

Compounds of Formula 2b are compounds wherein n is 1 and Y is C.

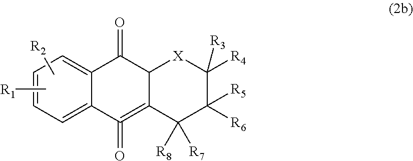

In the Formula 2a or 2b, R₁, R₂, R₃, R₄, R₅, R₆, R₇, R₈ and X are as defined in Formula 2.

Effective substance which exerts therapeutic effect on the treatment and/or prevention of prostate and/or testicle (seminal glands)-related diseases in the present invention is often referred to as “active ingredient” hereinafter.

### Preparation of Active Ingredient

In the pharmaceutical composition in accordance with the present invention, compounds of Formula 1 or Formula 2, as will be illustrated hereinafter, can be prepared by conventional methods known in the art and/or various processes which are based upon the general technologies and practices in the organic chemistry synthesis field. The preparation processes described below are only exemplary ones and other processes can also be employed. As such, the scope of the instant invention is not limited to the following processes.

In general, tricyclic naphthoquinone (pyrano-o-naphthoquinone and furano-o-naphthoquinone) derivatives can be synthesized by two methods mainly. One is to derive cyclization reaction using 3-allyl-2-hydroxy-1,4-naphthoquinone in acid catalyst condition, as the following β-lapachone synthesis scheme.

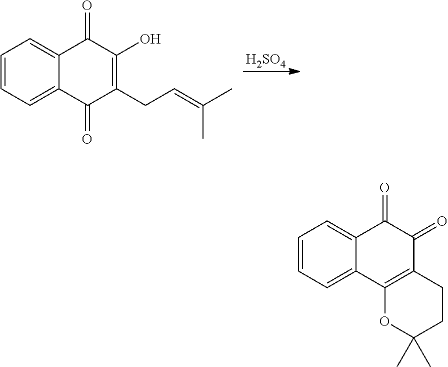

That is, 3-allyloxy-1,4-phenanthrenequinone can be obtained by deriving Diels-Alder reaction between 2-allyloxy-1,4-benzoquinone and styrene or 1-vinylcyclohexane derivatives and dehydrating the resulting intermediates using oxygen present in the air or oxidants such as NaIO₄ and DDQ. By further re-heating the above compound, 2-allyl-3-hydroxy-1,4-phenanthrenequinone of Lapachole form can be synthesized via Claisen rearrangement.

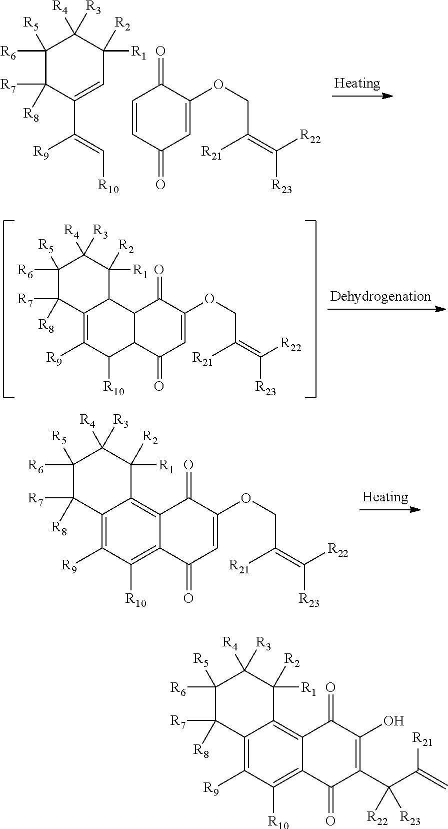

When the thus obtained 2-allyl-3-hydroxy-1,4-phenanthrenequinone is ultimately subjected to cyclization in an acid catalyst condition, various 3,4-phenanthrenequinone-based or 5,6,7,8-tetrahydro-3,4-phenanthrenequinone-based compounds can be synthesized. In this case, 5 or 6-cyclic cyclization occurs depending on the types of substituents (R₂₁, R₂₂, R₂₃ in the above formula) represented in the above formula, and also they are converted to the corresponding, adequate substituents (R₁₁, R₁₂, R₁₃, R₁₄, R₁₅, R₁₆ in the below formula).

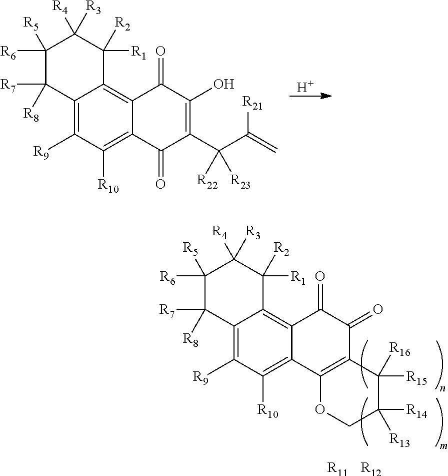

Further, 3-allyloxy-1,4-phenanthrenequinone is hydrolyzed to 3-oxy-1,4-phenanthrenequinone, in the condition of acid (H⁺) or alkali (OH⁻) catalyst, which is then reacted with various allyl halides to synthesize 2-allyl-3-hydroxy-1,4-phenanthrenequinone by C-alkylation. The thus obtained 2-allyl-3-hydroxy-1,4-phenanthrenequinone derivatives are subject to cyclization in the condition of acid catalyst to synthesize various 3,4-phenanthrenequinone-based or 5,6,7,8-tetrahydro-3,4-naphthoquinone-based compounds. In this case, 5 or 6-cyclic cyclization occurs depending on the types of substituents (R₂₁, R₂₂, R₂₃ in the above formula) represented in the above formula, and also they are converted to the corresponding, adequate substituents (R₁₁, R₁₂, R₁₃, R₁₄, R₁₅, R₁₆ in the below formula).

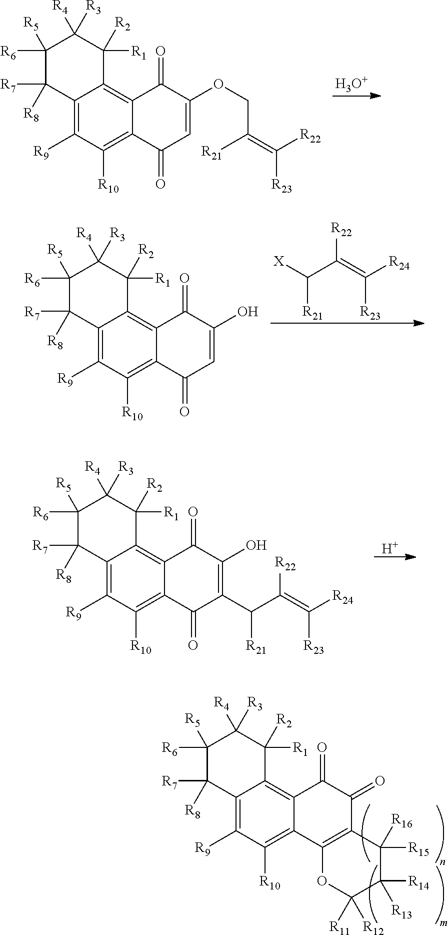

However, compounds in which substituents R₁₁ and R₁₂ are simultaneously hydrogen cannot be obtained by acid-catalyzed cyclization. These derivatives are obtained on the basis of a method reported by J. K. Snyder et al (Tetrahedron Letters 28 (1987), 3427-3430), more specifically, by first obtaining furanobenzoquinone introduced furan ring by cyclization, and then obtaining tricyclic phenanthroquinone by cyclization with 1-vinylcyclohexene derivatives, followed by reduction via hydrogen-addition. The above synthesis process can be summarized as follows.

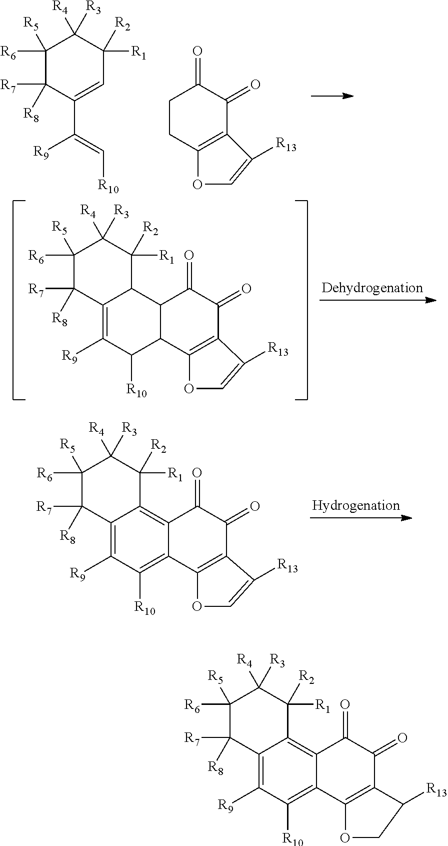

Besides the above synthetic method, compounds according to present invention in which substituents R₁₁ and R₁₂ are simultaneously hydrogen can be synthesized by the following method.

Preparation method 1 is a synthesis of active ingredient by acid-catalyzed cyclization which may be summarized in the general chemical reaction scheme as follows.

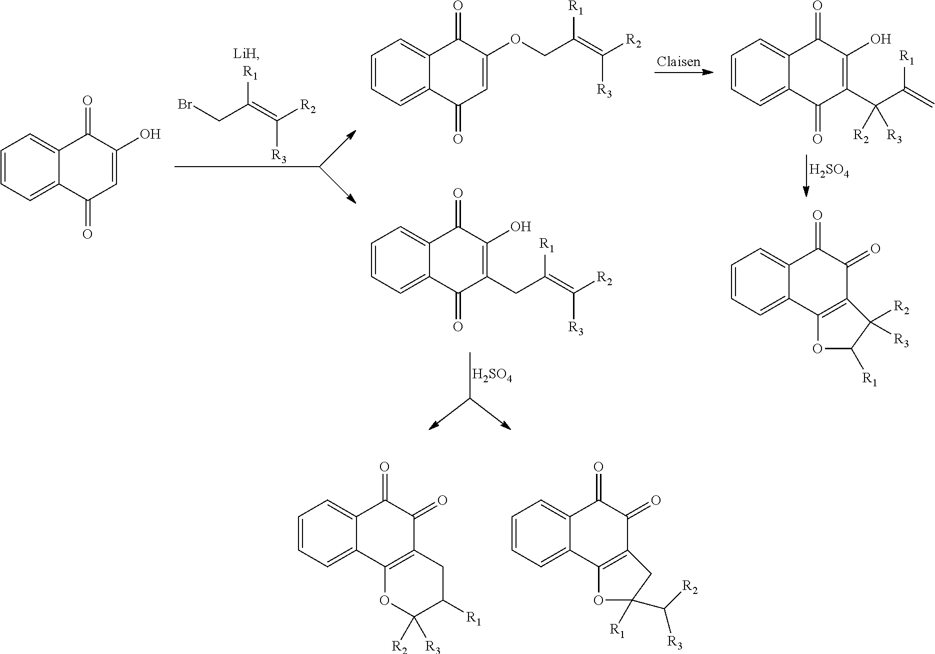

That is, when 2-hydroxy-1,4-naphthoquinone is reacted with various allylic bromides or equivalents thereof in the presence of a base, a C-alkylation product and an O-alkylation product are concurrently obtained. It is also possible to synthesize only either of two derivatives depending upon reaction conditions. Since O-alkylated derivative is converted into another type of C-alkylated derivative through Claisen Rearrangement by refluxing the O-alkylated derivative using a solvent such as toluene or xylene, it is possible to obtain various types of 3-substituted-2-hydroxy-1,4-naphthoquinone derivatives. The various types of C-alkylated derivatives thus obtained may be subjected to cyclization using sulfuric acid as a catalyst, thereby being capable of synthesizing pyrano-o-naphthoquinone or furano-o-naphthoquinone derivatives among the compounds.

Preparation method 2 is Diels-Alder reaction using 3-methylene-1,2,4-[3H]naphthalenetrione. As taught by V. Nair et al, Tetrahedron Lett. 42 (2001), 4549-4551, it is reported that a variety of pyrano-o-naphthoquinone derivatives can be relatively easily synthesized by subjecting 3-methylene-1,2,4-[3H]naphthalenetrione, produced upon heating 2-hydroxy-1,4-naphthoquinone and formaldehyde together, to Diels-Alder reaction with various olefin compounds. This method is advantageous in that various forms of pyrano-o-naphtho-quinone derivatives can be synthesized in a relatively simplified manner, as compared to induction of cyclization using sulfuric acid as a catalyst.

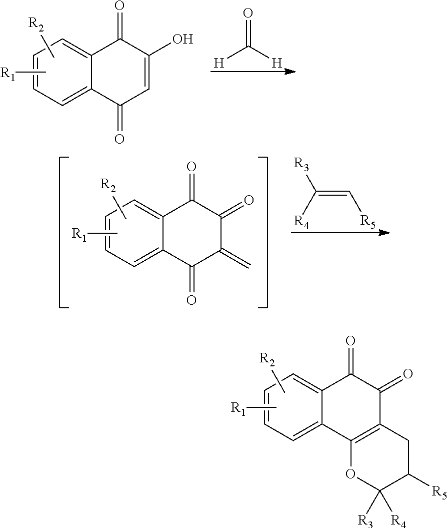

Preparation method 3 is haloakylation and cyclization by radical reaction. The same method used in synthesis of cryptotanshinone and 15,16-dihydro-tanshinone can also be conveniently employed for synthesis of furano-o-naphthoquinone derivatives. That is, as taught by A. C. Baillie et al (J. Chem. Soc. (C) 1968, 48-52), 2-haloethyl or 3-haloethyl radical chemical species, derived from 3-halopropanoic acid or 4-halobutanoic acid derivative, can be reacted with 2-hydroxy-1,4-naphthoquinone to thereby synthesize 3-(2-haloethyl or 3-halopropyl)-2-hydroxy-1,4-naphthoquinone, which is then subjected to cyclization under suitable acidic catalyst conditions to synthesize various pyrano-o-naphthoquinone or furano-o-naphthoquinone derivatives.

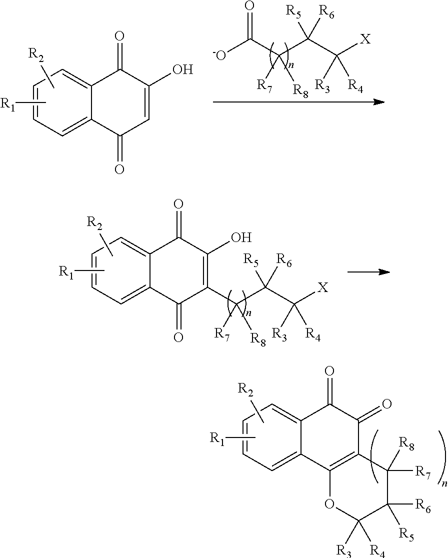

Preparation method 4 is cyclization of 4,5-benzofurandione by Diels-Alder reaction. Another method used in synthesis of cryptotanshinone and 15,16-dihydro-tanshinone may be a method taught by J. K. Snyder et al (Tetrahedron Letters 28 (1987), 3427-3430). According to this method, furano-o-naphthoquinone derivatives can be synthesized by cycloaddition via Diels-Alder reaction between 4,5-benzofurandione derivatives and various diene derivatives.

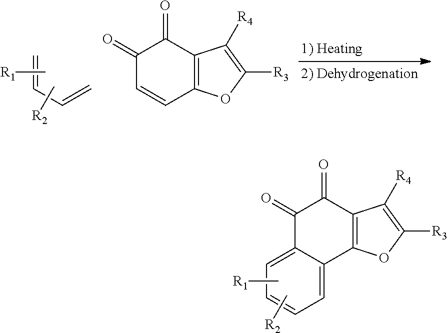

Based on the above-mentioned preparation methods, various derivatives may be synthesized using relevant synthesis methods, depending upon kinds of substituents.

Among compounds of according to the present invention, particularly preferred are in Table 1 below, but are not limited thereto.

TABLE 1

| | | | | Preparation |
|---|---|---|---|---|
| No. | Chemical structure | Formula | Molecular weight | method |
| 1 | 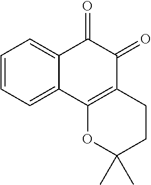 | C₁₅H₁₄O₃ | 242.27 | Method 1 |
| 2 | 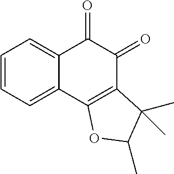 | C₁₅H₁₄O₃ | 242.27 | Method 1 |
| 3 | 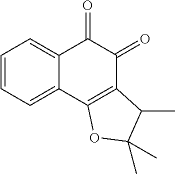 | C₁₅H₁₄O₃ | 242.27 | Method 1 |
| 4 | 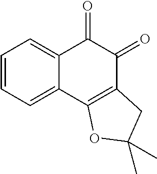 | C₁₄H₁₂O₃ | 228.24 | Method 1 |
| 5 | 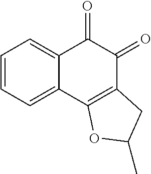 | C₁₃H₁₀O₃ | 214.22 | Method 1 |
| 6 | 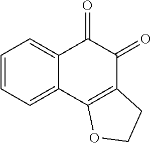 | C₁₂H₈O₃ | 200.19 | Method 2 |
| 7 | 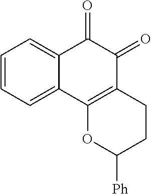 | C₁₉H₁₄O₃ | 290.31 | Method 1 |
| 8 | 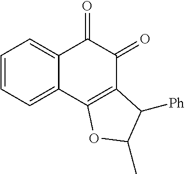 | C₁₉H₁₄O₃ | 290.31 | Method 1 |
| 9 | 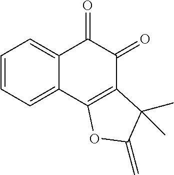 | C₁₅H₁₂O₃ | 240.25 | Method 1 |
| 10 | 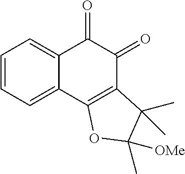 | C₁₆H₁₆O₄ | 272.30 | Method 1 |
| 11 | 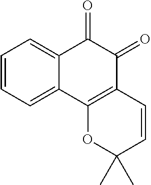 | C₁₅H₁₂O₃ | 240.25 | Method 1 |
| 12 | 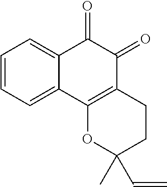 | C₁₆H₁₄O₃ | 254.28 | Method 2 |
| 13 | 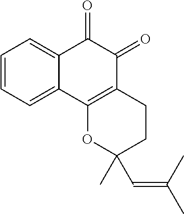 | C₁₈H₁₈O₃ | 282.33 | Method 2 |
| 14 | 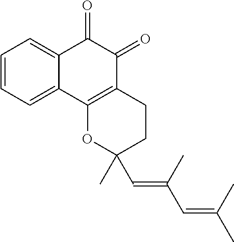 | C₂₁H₂₂O₃ | 322.40 | Method 2 |
| 15 | 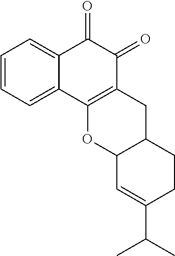 | C₂₁H₂₂O₃ | 322.40 | Method 2 |
| 16 | 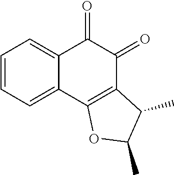 | C₁₄H₁₂O₃ | 228.24 | Method 1 |
| 17 | 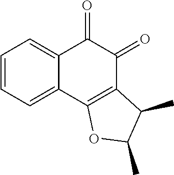 | C₁₄H₁₂O₃ | 228.24 | Method 1 |
| 18 | 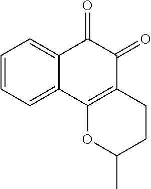 | C₁₄H₁₂O₃ | 228.24 | Method 1 |
| 19 | 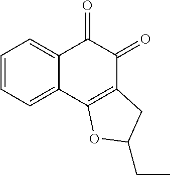 | C₁₄H₁₂O₃ | 228.24 | Method 1 |
| 20 | 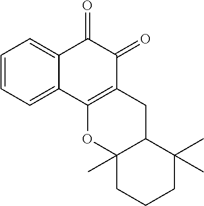 | C₂₀H₂₂O₃ | 310.39 | Method 1 |
| 21 | 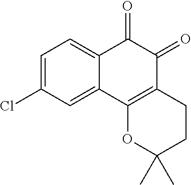 | C₁₅H₁₃ClO₃ | 276.71 | Method 1 |
| 22 | 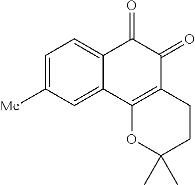 | C₁₆H₁₆O₃ | 256.30 | Method 1 |
| 23 | 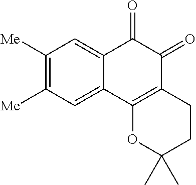 | C₁₇H₁₈O₅ | 302.32 | Method 1 |
| 24 | 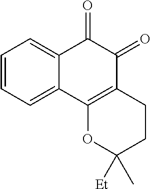 | C₁₆H₁₆O₃ | 256.30 | Method 1 |
| 25 | 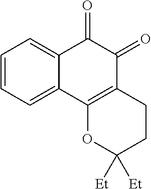 | C₁₇H₁₈O₃ | 270.32 | Method 1 |
| 26 | 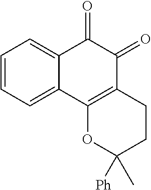 | C₂₀H₁₆O₃ | 304.34 | Method 1 |
| 27 | 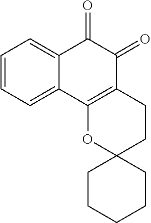 | C₁₈H₁₈O₃ | 282.33 | Method 1 |
| 28 | 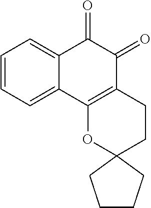 | C₁₇H₁₆O₃ | 268.31 | Method 1 |
| 29 | 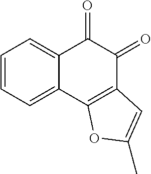 | C₁₃H₈O₃ | 212.20 | Method 1 |
| 30 | 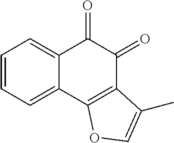 | C₁₃H₈O₃ | 212.20 | Method 4 |
| 31 | 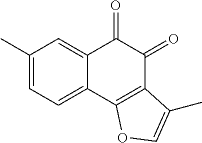 | C₁₄H₁₀O₃ | 226.23 | Method 4 |
| 32 | 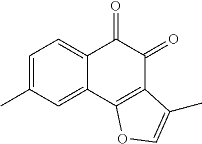 | C₁₄H₁₀O₃ | 226.23 | Method 4 |
| 33 | 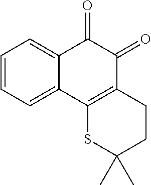 | C₁₅H₁₄O₂S | 258.34 | Method 1 |
| 34 | 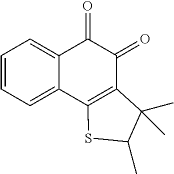 | C₁₅H₁₄O₂S | 258.34 | Method 1 |
| 35 | 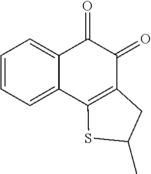 | C₁₃H₁₀O₂S | 230.28 | Method 1 |
| 36 | 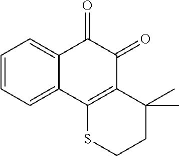 | C₁₅H₁₄O₂S | 258.34 | Method 2 |
| 37 | 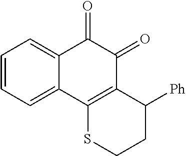 | C₁₉H₁₄O₂S | 306.38 | Method 2 |
| 38 | 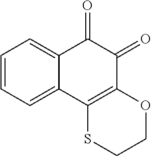 | C₁₂H₈O₃S | 232.26 | Method 3 |
| 39 | 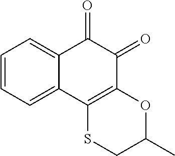 | C₁₃H₁₀O₃S | 246.28 | Method 3 |
| 40 | 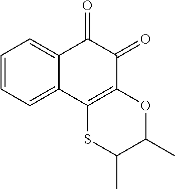 | C₁₄H₁₂O₃S | 260.31 | Method 3 |
| 41 | 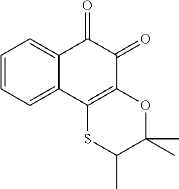 | C₁₅H₁₄O₃S | 274.34 | Method 3 |
| 42 | 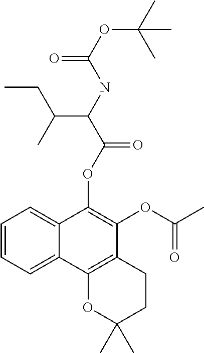 | C₂₈H₃₇O₇N | 502.22 | — |
| 43 | 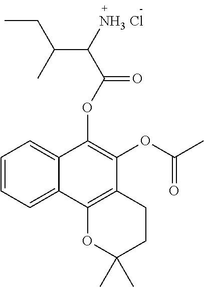 | C₂₃H₃₀O₅NCl | 940.32 | — |
| 44 | 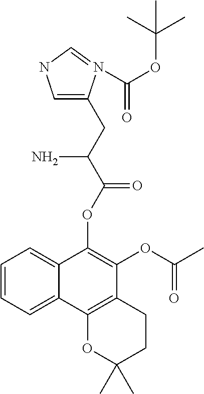 | C₂₈H₃₃O₇N₃ | 526.22 | — |
| 45 | 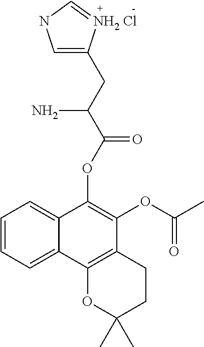 | C₂₃H₂₆O₅N₃Cl | 988.32 | — |
| 46 | 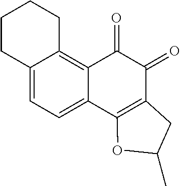 | C₁₇H₁₆O₃ | 268.31 | — |
| 47 | 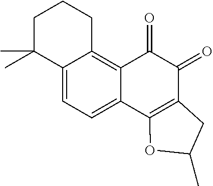 | C₁₉H₂₀O₃ | 296.36 | — |
| 48 | 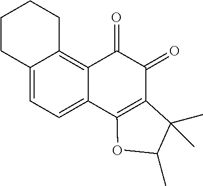 | C₁₉H₂₀O₃ | 296.36 | — |
| 49 | 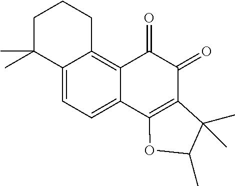 | C₂₁H₂₄O₃ | 324.41 | — |
| 50 | 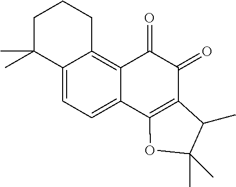 | C₂₁H₂₄O₃ | 324.41 | — |
| 51 | 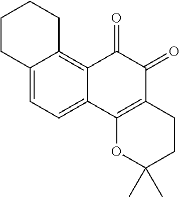 | C₁₉H₂₀O₃ | 296.36 | — |
| 52 | 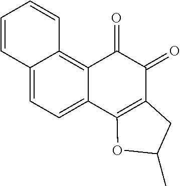 | C₁₇H₁₂O₃ | 264.28 | — |
| 53 | 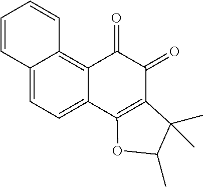 | C₁₉H₁₆O₃ | 292.33 | — |
| 54 | 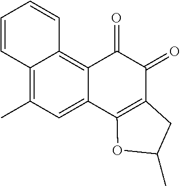 | C₁₈H₁₄O₃ | 278.30 | — |
| 55 | 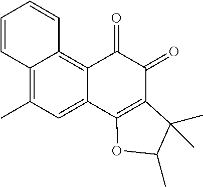 | C₂₀H₁₈O₃ | 306.36 | — |
| 56 | 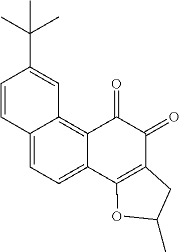 | C₂₁H₂₀O₃ | 320.38 | — |
| 57 | 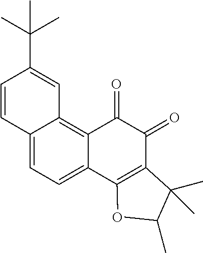 | C₂₃H₂₄O₃ | 348.43 | — |
| 58 | 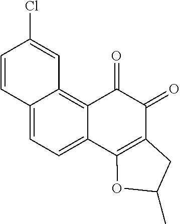 | C₁₇H₁₁ClO₃ | 298.72 | — |
| 59 | 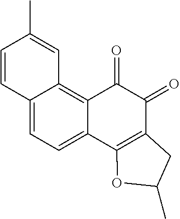 | C₁₈H₁₄O₃ | 278.30 | — |
| 60 | 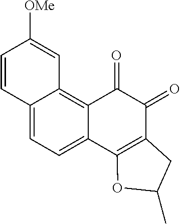 | C₁₈H₁₄O₄ | 294.30 | — |
| 61 | 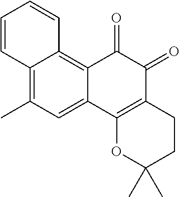 | C₂₀H₁₈O₃ | 306.36 | — |
| 62 | 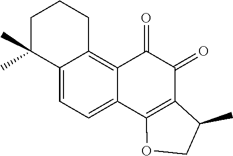 | C₁₈H₁₈O₃ | 282.33 | — |
| 63 | 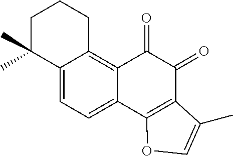 | C₁₈H₁₆O₃ | 280.33 | — |
| 64 | 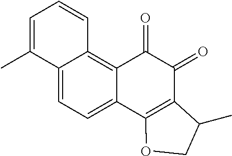 | C₁₈H₁₄O₃ | 278.33 | — |
| 65 | 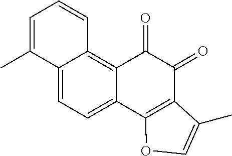 | C₁₈H₁₂O₃ | 276.33 | — |

The term “pharmaceutical composition” as used herein means a mixture of the compound of Formula 1 or 2 with other chemical components, such as diluents or carriers. The pharmaceutical composition facilitates administration of the compound to an organism. Various techniques of administering a compound are known in the art and include, but are not limited to oral, injection, aerosol, parenteral and topical administrations. Pharmaceutical compositions can also be obtained by reacting compounds of interest with acids such as hydrochloric acid, hydrobromic acid, sulfuric acid, nitric acid, phosphoric acid, methanesulfonic acid, p-toluenesulfonic acid, salicylic acid and the like. The effective ingredients, therapeutically effective for the treatment and prevention of restenosis include all the compounds of Formula in the above, referring “active ingredient” hereafter.

The term “therapeutically effective amount” means an amount of an active ingredient that is effective to relieve or reduce to some extent one or more of the symptoms of the disease in need of treatment, or to retard initiation of clinical markers or symptoms of a disease in need of prevention, when the compound is administered. Thus, a therapeutically effective amount refers to an amount of the active ingredient which exhibit effects of (i) reversing the rate of progress of a disease; (ii) inhibiting to some extent further progress of the disease; and/or, (iii) relieving to some extent (or, preferably, eliminating) one or more symptoms associated with the disease. The therapeutically effective amount may be empirically determined by experimenting with the compounds concerned in known in vivo and in vitro model systems for a disease in need of treatment.

In the pharmaceutical composition in accordance with the present invention, compounds of Formula 1 or 2 as an active ingredient, as will be illustrated hereinafter, can be prepared by conventional methods known in the art and/or various processes which are based upon the general technologies and practices in the organic chemistry synthesis field.

The pharmaceutical composition of the present invention may be manufactured in a manner that is itself known, e.g., by means of conventional mixing, dissolving, granulating, dragee-making, levigating, emulsifying, encapsulating, entrapping or lyophilizing processes.

Therefore, pharmaceutical compositions for use in accordance with the present invention may be additionally comprised of a pharmaceutically acceptable carrier, a diluent or an excipient, or any combination thereof. That may be formulated in a conventional manner using one or more pharmaceutically acceptable carriers comprising excipients and auxiliaries which facilitate processing of the active compounds into preparations which can be used pharmaceutically. The pharmaceutical composition facilitates administration of the compound to an organism.

The term “carrier” means a chemical compound that facilitates the incorporation of a compound into cells or tissues. For example, dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffer solution is phosphate buffered saline (PBS) because it mimics the ionic strength conditions of human body fluid. Since buffer salts can control the pH of a solution at low concentrations, a buffer diluent rarely modifies the biological activity of a compound.

The compounds described herein may be administered to a human patient per se, or in the form of pharmaceutical compositions in which they are mixed with other active ingredients, as in combination therapy, or suitable carriers or excipient(s). Proper formulation is dependent upon the route of administration chosen. Techniques for formulation and administration of the compounds may be found in “Remington's Pharmaceutical Sciences,” Mack Publishing Co., Easton, Pa., 18th edition, 1990.

Various techniques relating to pharmaceutical formulation for administering an active ingredient into the body are known in the art and include, but are not limited to oral, injection, aerosol, parenteral and topical administrations. If necessary, they can also be obtained by reacting compounds of interest with acids such as hydrochloric acid, hydrobromic acid, sulfuric acid, nitric acid, phosphoric acid, methanesulfonic acid, p-toluenesulfonic acid, salicylic acid and the like.

Pharmaceutical formulation may be carried out by conventional methods known in the art and, preferably, the pharmaceutical formulation may be oral, external, transdermal, transmucosal and an injection formulation, and particularly preferred is oral formulation.

Meanwhile, for injection, the agents of the present invention may be formulated in aqueous solutions, preferably in physiologically compatible buffers such as Hanks's solution, Ringer's solution, or physiological saline. For transmucosal administration, penetrants appropriate to the barrier to be permeated are used in the formulation. Such penetrants are generally known in the art.

The pharmaceutical compounds in accordance with the present invention, may be particularly preferably an oral pharmaceutical composition which is prepared into an intestine-targeted formulation.

Generally, an oral pharmaceutical composition passes through the stomach upon oral administration, is largely absorbed by the small intestine and then diffused into all the tissues of the body, thereby exerting therapeutic effects on the target tissues.

In this connection, the oral pharmaceutical composition according to the present invention enhances bioabsorption and bioavailability of a compound of Formula 1 or Formula 2 active ingredient via intestine-targeted formulation of the active ingredient. More specifically, when the active ingredient in the pharmaceutical composition according to the present invention is primarily absorbed in the stomach, and upper parts of the small intestine, the active ingredient absorbed into the body directly undergoes liver metabolism which is then accompanied by substantial degradation of the active ingredient, so it is impossible to exert a desired level of therapeutic effects. On the other hand, it is expected that when the active ingredient is largely absorbed around and downstream of the lower small intestine, the absorbed active ingredient migrates via lymph vessels to the target tissues to thereby exert high therapeutic effects.

Further, as it is constructed in such a way that the pharmaceutical composition according to the present invention targets up to the colon which is a final destination of the digestion process, it is possible to increase the in vivo retention time of the drug and it is also possible to minimize decomposition of the drug which may take place due to the body metabolism upon administration of the drug into the body. As a result, it is possible to improve pharmacokinetic properties of the drug, to significantly lower a critical effective dose of the active ingredient necessary for the treatment of the disease, and to obtain desired therapeutic effects even with administration of a trace amount of the active ingredient. Further, in the oral pharmaceutical composition, it is also possible to minimize the absorption variation of the drug by reducing the between- and within-individual variation of the bioavailability which may result from intragastric pH changes and dietary uptake patterns.

Therefore, the intestine-targeted formulation according to the present invention is configured such that the active ingredient is largely absorbed in the small and large intestines, more preferably in the jejunum, and the ileum and colon corresponding to the lower small intestine, particularly preferably in the ileum or colon.

The intestine-targeted formulation may be designed by taking advantage of numerous physiological parameters of the digestive tract, through a variety of methods. In one preferred embodiment of the present invention, the intestine-targeted formulation may be prepared by (1) a formulation method based on a pH-sensitive polymer, (2) a formulation method based on a biodegradable polymer which is decomposable by an intestine-specific bacterial enzyme, (3) a formulation method based on a biodegradable matrix which is decomposable by an intestine-specific bacterial enzyme, or (4) a formulation method which allows release of a drug after a given lag time, and any combination thereof.

Specifically, the intestine-targeted formulation (1) using the pH-sensitive polymer is a drug delivery system which is based on pH changes of the digestive tract. The pH of the stomach is in a range of 1 to 3, whereas the pH of the small and large intestines has a value of 7 or higher, as compared to that of the stomach. Based on this fact, the pH-sensitive polymer may be used in order to ensure that the pharmaceutical composition reaches the lower intestinal parts without being affected by pH fluctuations of the digestive tract. Examples of the pH-sensitive polymer may include, but are not limited to, at least one selected from the group consisting of methacrylic acid-ethyl acrylate copolymer (Eudragit: Registered Trademark of Rohm Pharma GmbH), hydroxypropylmethyl cellulose phthalate (HPMCP) and a mixture thereof.

Preferably, the pH-sensitive polymer may be added by a coating process. For example, addition of the polymer may be carried out by mixing the polymer in a solvent to form an aqueous coating suspension, spraying the resulting coating suspension to form a film coating, and drying the film coating.

The intestine-targeted formulation (2) using the biodegradable polymer which is decomposable by the intestine-specific bacterial enzyme is based on the utilization of a degradative ability of a specific enzyme that can be produced by enteric bacteria. Examples of the specific enzyme may include azoreductase, bacterial hydrolase glycosidase, esterase, polysaccharidase, and the like.

When it is desired to design the intestine-targeted formulation using azoreductase as a target, the biodegradable polymer may be a polymer containing an azoaromatic linkage, for example, a copolymer of styrene and hydroxyethylmethacrylate (HEMA). When the polymer is added to the formulation containing the active ingredient, the active ingredient may be liberated into the intestine by reduction of an azo group of the polymer via the action of the azoreductase which is specifically secreted by enteric bacteria, for example, Bacteroides fragilis and Eubacterium limosum.

When it is desired to design the intestine-targeted formulation using glycosidase, esterase, or polysaccharidase as a target, the biodegradable polymer may be a naturally-occurring polysaccharide or a substituted derivative thereof. For example, the biodegradable polymer may be at least one selected from the group consisting of dextran ester, pectin, amylose, ethyl cellulose and a pharmaceutically acceptable salt thereof. When the polymer is added to the active ingredient, the active ingredient may be liberated into the intestine by hydrolysis of the polymer via the action of each enzyme which is specifically secreted by enteric bacteria, for example, Bifidobacteria and Bacteroides spp. These polymers are natural materials, and have an advantage of low risk of in vivo toxicity.

The intestine-targeted formulation (3) using the biodegradable matrix which is decomposable by an intestine-specific bacterial enzyme may be a form in which the biodegradable polymers are cross-linked to each other and are added to the active ingredient or the active ingredient-containing formulation. Examples of the biodegradable polymer may include naturally-occurring polymers such as chondroitin sulfate, guar gum, chitosan, pectin, and the like. The degree of drug release may vary depending upon the degree of cross-linking of the matrix-constituting polymer.

In addition to the naturally-occurring polymers, the biodegradable matrix may be a synthetic hydrogel based on N-substituted acrylamide. For example, there may be used a hydrogel synthesized by cross-linking of N-tert-butylacryl amide with acrylic acid or copolymerization of 2-hydroxyethyl methacrylate and 4-methacryloyloxyazobenzene, as the matrix. The cross-linking may be, for example an azo linkage as mentioned above, and the formulation may be a form where the density of cross-linking is maintained to provide the optimal conditions for intestinal drug delivery and the linkage is degraded to interact with the intestinal mucous membrane when the drug is delivered to the intestine.

Further, the intestine-targeted formulation (4) with time-course release of the drug after a lag time is a drug delivery system utilizing a mechanism that is allowed to release the active ingredient after a predetermined time irrespective of pH changes. In order to achieve enteric release of the active drug, the formulation should be resistant to the gastric pH environment, and should be in a silent phase for 5 to 6 hours corresponding to a time period taken for delivery of the drug from the body to the intestine, prior to release of the active ingredient into the intestine. The time-specific delayed-release formulation may be prepared by addition of the hydrogel prepared from copolymerization of polyethylene oxide with polyurethane.

Specifically, the delayed-release formulation may have a configuration in which the formulation absorbs water and then swells while it stays within the stomach and the upper digestive tract of the small intestine, upon addition of a hydrogel having the above-mentioned composition after applying the drug to an insoluble polymer, and then migrates to the lower part of the small intestine which is the lower digestive tract and liberates the drug, and the lag time of drug is determined depending upon a length of the hydrogel.

As another example of the polymer, ethyl cellulose (EC) may be used in the delayed-release dosage formulation. EC is an insoluble polymer, and may serve as a factor to delay a drug release time, in response to swelling of a swelling medium due to water penetration or changes in the internal pressure of the intestines due to a peristaltic motion. The lag time may be controlled by the thickness of EC. As an additional example, hydroxypropylmethyl cellulose (HPMC) may also be used as a retarding agent that allows drug release after a given period of time by thickness control of the polymer, and may have a lag time of 5 to 10 hours.

In the oral pharmaceutical composition according to the present invention, the active ingredient may have a crystalline structure with a high degree of crystallinity, or a crystalline structure with a low degree of crystallinity.

As used herein, the term “degree of crystallinity” is defined as the weight fraction of the crystalline portion of the total crystalline compound and may be determined by a conventional method known in the art. For example, measurement of the degree of crystallinity may be carried out by a density method or precipitation method which calculates the crystallinity degree by previous assumption of a preset value obtained by addition and/or reduction of appropriate values to/from each density of the crystalline portion and the amorphous portion, a method involving measurement of the heat of fusion, an X-ray method in which the crystallinity degree is calculated by separation of the crystalline diffraction fraction and the noncrystalline diffraction fraction from X-ray diffraction intensity distribution upon X-ray diffraction analysis, or an infrared method which calculates the crystallinity degree from a peak of the width between crystalline bands of the infrared absorption spectrum.

In the oral pharmaceutical composition according to the present invention, the crystallinity degree of the active ingredient is preferably 50% or less. More preferably, the active ingredient may have an amorphous structure from which the intrinsic crystallinity of the material was completely lost. The amorphous compound exhibits a relatively high solubility, as compared to the crystalline compound, and can significantly improve a dissolution rate and in vivo absorption rate of the drug.

In one preferred embodiment of the present invention, the amorphous structure may be formed during preparation of the active ingredient into microparticles or fine particles (micronization of the active ingredient). The microparticles may be prepared, for example by spray drying of active ingredients, melting methods involving formation of melts of active ingredients with polymers, co-precipitation involving formation of co-precipitates of active ingredients with polymers after dissolution of active ingredients in solvents, inclusion body formation, solvent volatilization, and the like. Preferred is spray drying. Even when the active ingredient is not of an amorphous structure, that is, has a crystalline structure or semi-crystalline structure, micronization of the active ingredient into fine particles via mechanical milling contributes to improvement of solubility, due to a large specific surface area of the particles, consequently resulting in improved dissolution rate and bioabsorption rate of the active drug.

The spray drying is a method of making fine particles by dissolving the active ingredient in a certain solvent and the spray-drying the resulting solution. During the spray-drying process, a high percent of the crystallinity of the naphthoquinone compound is lost to thereby result in an amorphous state, and therefore the spray-dried product in the form of a fine powder is obtained.

The mechanical milling is a method of grinding the active ingredient into fine particles by applying strong physical force to active ingredient particles. The mechanical milling may be carried out by using a variety of milling processes such as jet milling, ball milling, vibration milling, hammer milling, and the like. Particularly preferred is jet milling which can be carried out using an air pressure, at a temperature of less than 40° C.

Meanwhile, irrespective of the crystalline structure, a decreasing particle diameter of the particulate active ingredient leads to an increasing specific surface area, thereby increasing the dissolution rate and solubility. However, an excessively small particle diameter makes it difficult to prepare fine particles having such a size and also brings about agglomeration or aggregation of particles which may result in deterioration of the solubility. Therefore, in one preferred embodiment, the particle diameter of the active ingredient may be in a range of 5 nm to 500 μm. In this range, the particle agglomeration or aggregation can be maximally inhibited, and the dissolution rate and solubility can be maximized due to a high specific surface area of the particles.

Preferably, a surfactant may be additionally added to prevent the particle agglomeration or aggregation which may occur during formation of the fine particles, and/or an antistatic agent may be additionally added to prevent the occurrence of static electricity.

If necessary, a moisture-absorbent material may be further added during the milling process. The compound of Formula 1 or Formula 2 has a tendency to be crystallized by water, so incorporation of the moisture-absorbent material inhibits recrystallization of the naphthoquinone-based compound over time and enables maintenance of increased solubility of compound particles due to micronization. Further, the moisture-absorbent material serves to suppress coagulation and aggregation of the pharmaceutical composition while not adversely affecting therapeutic effects of the active ingredient.

Examples of the surfactant may include, but are not limited to, anionc surfactants such as docusate sodium and sodium lauryl sulfate; cationic surfactants such as benzalkonium chloride, benzethonium chloride and cetrimide; nonionic surfactants such as glyceryl monooleate, polyoxyethylene sorbitan fatty acid ester, and sorbitan ester; amphiphilic polymers such as polyethylene-polypropylene polymer and polyoxyethylene-polyoxypropylene polymer (Poloxamer), and Gelucire™ series (Gattefosse Corporation, USA); propylene glycol monocaprylate, oleoyl macrogol-6-glyceride, linoleoyl macrogol-6-glyceride, caprylocaproyl macrogol-8-glyceride, propylene glycol monolaurate, and polyglyceryl-6-dioleate. These materials may be used alone or in any combination thereof.

Examples of the moisture-absorbent material may include, but are not limited to, colloidal silica, light anhydrous silicic acid, heavy anhydrous silicic acid, sodium chloride, calcium silicate, potassium aluminosilicate, calcium aluminosilicate, and the like. These materials may be used alone or in any combination thereof.

Some of the above-mentioned moisture absorbents may also be used as the antistatic agent.

The surfactant, antistatic agent, and moisture absorbent are added in a certain amount that is capable of achieving the above-mentioned effects, and such an amount may be appropriately adjusted depending upon micronization conditions. Preferably, the additives may be used in a range of 0.05 to 20% by weight, based on the total weight of the active ingredient.

In one preferred embodiment, during formulation of the pharmaceutical composition according to the present invention into preparations for oral administration, water-soluble polymers, solubilizers and disintegration-promoting agents may be further added. Preferably, formulation of the composition into a desired dosage form may be made by mixing the additives and the particulate active ingredient in a solvent and spray-drying the mixture.

The water-soluble polymer is of help to prevent aggregation of the particulate active ingredients, by rendering surroundings of naphthoquinone-based compound molecules or particles hydrophilic to consequently enhance water solubility, and preferably to maintain the amorphous state of the active ingredient compound of Formula 1 or Formula 2.

Preferably, the water-soluble polymer is a pH-independent polymer, and can bring about crystallinity loss and enhanced hydrophilicity of the active ingredient, even under the between- and within-individual variation of the gastrointestinal pH.

Preferred examples of the water-soluble polymers may include at least one selected from the group consisting of cellulose derivatives such as methyl cellulose, hydroxymethyl cellulose, hydroxyethyl cellulose, ethyl cellulose, hydroxyethylmethyl cellulose, carboxymethyl cellulose, hydroxypropylmethyl cellulose, hydroxypropylmethyl cellulose phthalate, sodium carboxymethyl cellulose, and carboxymethylethyl cellulose; polyvinyl alcohols; polyvinyl acetate, polyvinyl acetate phthalate, polyvinylpyrrolidone (PVP), and polymers containing the same; polyalkene oxide or polyalkene glycol, and polymers containing the same. Preferred is hydroxypropylmethyl cellulose.

In the pharmaceutical composition of the present invention, an excessive content of the water-soluble polymer which is higher than a given level provides no further increased solubility, but disadvantageously brings about various problems such as overall increases in the hardness of the formulation, and non-penetration of an eluent into the formulation, by formation of films around the formulation due to excessive swelling of water-soluble polymers upon exposure to the eluent. Accordingly, the solubilizer is preferably added to maximize the solubility of the formulation by modifying physical properties of the compound of Formula 1 or Formula 2.

In this respect, the solubilizer serves to enhance solubilization and wettability of the sparingly-soluble compound of Formula 1 or Formula 2, and can significantly reduce the bioavailability variation of the naphthoquinone-based compound originating from diets and the time difference of drug administration after dietary uptake. The solubilizer may be selected from conventionally widely used surfactants or amphiphiles, and specific examples of the solubilizer may refer to the surfactants as defined above.

The disintegration-promoting agent serves to improve the drug release rate, and enables rapid release of the drug at the target site to thereby increase bioavailability of the drug.

Preferred examples of the disintegration-promoting agent may include, but are not limited to, at least one selected from the group consisting of Croscarmellose sodium, Crospovidone, calcium carboxymethylcellulose, starch glycolate sodium and lower substituted hydroxypropyl cellulose. Preferred is Croscarmellose sodium.

Upon taking into consideration various factors as described above, it is preferred to add 10 to 1000 parts by weight of the water-soluble polymer, 1 to 30 parts by weight of the disintegration-promoting agent and 0.1 to 20 parts by weight of the solubilizer, based on 100 parts by weight of the active ingredient.

In addition to the above-mentioned ingredients, other materials known in the art in connection with formulation may be optionally added, if necessary.

The solvent for spray drying is a material exhibiting a high solubility without modification of physical properties thereof and easy volatility during the spray drying process. Preferred examples of such a solvent may include, but are not limited to, dichloromethane, chloroform, methanol, and ethanol. These materials may be used alone or in any combination thereof. Preferably, a content of solids in the spray solution is in a range of 5 to 50% by weight, based on the total weight of the spray solution.

The above-mentioned intestine-targeted formulation process may be preferably carried out for formulation particles prepared as above.

In one preferred embodiment, the oral pharmaceutical composition according to the present invention may be formulated by a process comprising the following steps:

(a) adding the compound of Formula 1 or Formula 2 alone or in combination with a surfactant and a moisture-absorbent material, and grinding the compound of Formula 1 with a jet mill to prepare active ingredient microparticles;

(b) dissolving the active ingredient microparticles in conjunction with a water-soluble polymer, a solubilizer and a disintegration-promoting agent in a solvent and spray-drying the resulting solution to prepare formulation particles; and

(c) dissolving the formulation particles in conjunction with a pH-sensitive polymer and a plasticizer in a solvent and spray-drying the resulting solution to carry out intestine-targeted coating on the formulation particles.

The surfactant, moisture-absorbent material, water-soluble polymer, solubilizer and disintegration-promoting agent are as defined above. The plasticizer is an additive added to prevent hardening of the coating, and may include, for example polymers such as polyethylene glycol.

Alternatively, formulation of the active ingredient may be carried out by sequential or concurrent spraying of vehicles of step (b) and intestine-targeted coating materials of step (c) onto jet-milled active ingredient particles of step (a) as a seed.

Pharmaceutical compositions suitable for use in the present invention include compositions in which the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

When the pharmaceutical composition of the present invention is formulated into a unit dosage form, the compound of Formula 1 or Formula 2 as the active ingredient is preferably contained in a unit dose of about 0.1 to 1,000 mg. The amount of the compound of Formula 1 or Formula 2 administered will be determined by the attending physician, depending upon body weight and age of patients being treated, characteristic nature and the severity of diseases. However, it is general that the amount of administration necessary for treatment of adult is in the range of about 1 to 3000 mg per day depending upon the frequency and intensity of administration. Generally, about 1 to 500 mg per day as a total administration amount is sufficient for the intramuscular or intravenous administration to adult; however, more administration amount would be desired for some patients.

In accordance with another aspect of the present invention, there is provided a use of a compound of Formula 1 or 2 in the preparation of a medicament for the treatment and prevention of cardiac diseases.

Examples of the cardiac diseases may include heart hypertrophy, heart failure, congestive heart failure, angina pectoris, myocardial infarction, etc. Preferred is heart hypertrophy or heart failure.

The term “treatment” means ceasing or delaying progress of diseases when the compounds of Formula 1 or 2 or compositions comprising the same are administered to subjects exhibiting symptoms of diseases. The term “prevention” means ceasing or delaying symptoms of diseases when the compounds of Formula 1 or 2 or compositions comprising the same are administered to subjects exhibiting no symptoms of diseases, but having high risk of developing symptoms of diseases.

### BRIEF DESCRIPTION OF THE DRAWINGS

FIGS. 1 to 3 are graphs showing the results of HW/BW ratios, HW/TL ratios and fractional shortenings as measured in a heart hypertrophy model according to Experimental Example 1 (FIG. 1: HW/BW ratio, FIG. 2: HW/TL ratio, and FIG. 3: fractional shortening);

FIGS. 4 to 6 are graphs showing the results of HW/BW ratios, HW/TL ratios and fractional shortenings as measured in a heart failure model according to Experimental Example 1 (FIG. 4: HW/BW ratio, FIG. 5: HW/TL ratio, and FIG. 6: fractional shortening);

FIGS. 7 to 9 are graphs showing the results of HW/BW ratios, HW/TL ratios and fractional shortenings as measured in a heart hypertrophy model according to Experimental Example 2 (FIG. 7: HW/BW ratio, FIG. 8: HW/TL ratio, and FIG. 9: fractional shortening);

FIGS. 10 to 12 are graphs showing the results of HW/BW ratios, HW/TL ratios and fractional shortenings as measured in a heart failure model according to Experimental Example 2 (FIG. 10: HW/BW ratio, FIG. 11: HW/TL ratio, and FIG. 12: fractional shortening);

FIG. 13 is a graph showing time-course changes in body weight and dietary intake in a heart hypertrophy model according to Experimental Example 3;

FIG. 14 is a graph showing a HW/BW ratio in response to a dose of MB660 as measured in Experimental Example 3;

FIG. 15 is a micrograph showing changes of a heart size in a heart hypertrophy model according to Experimental Example 4;

FIG. 16 is a micrograph showing mitochondrial changes of cardiomyocytes in response to the administration of MB660 in a heart hypertrophy model according to Experimental Example 5; and

FIG. 17 is a micrograph showing mitochondrial changes of cardiomyocytes in response to the administration of MB660 in a heart failure model according to Experimental Example 5.

### DETAILED DESCRIPTION OF PREFERRED EMBODIMENTS

Now, the present invention will be described in more detail with reference to the following Examples. These examples are provided only for illustrating the present invention and should not be construed as limiting the scope and spirit of the present invention. Therapeutic effects of the pharmaceutical composition in accordance with the present invention will be confirmed as follows.

### Materials and Methods

### 1. Animal Models

8-week-old C57BL/6J male mice (n=40, 20-23 g, SLC, Japan) were purchased and subjected to transverse aortic constriction (TAC).

For the operation of TAC, C57BL/6 male mice were anesthetized with an intraperitoneal injection of a liquid anesthetic (ketamine:xylazine=20:1, kg/ml), and a 22-gauge IV catheter needle was placed into the trachea, followed by connection with a ventilator (Harvard Apparatus) for artificial forcible respiration. The thorax was incised and the thymus was removed. Following the removal of the thymus, the transverse aorta between the right subclavian artery and the left subclavian artery was banded (7-0, silk) with an overlaying blunted 27-gauge needle, and then the needle was quickly removed to create a defined constriction. The thorax was sutured with (5-0) silk to complete the surgery, and respiration of animals was then confirmed.

2 weeks after the operation of TAC for the heart hypertrophy model, a ratio of heart weight (HW) to tibia length (HW/TL ratio) was measured to confirm the degree of heart hypertrophy vs. control group, and it was determined whether induction of heart hypertrophy was appropriately made. 5 weeks after the operation of TAC for the heart failure model, a difference of a fractional shortening (F.S) value (%) with the control group was confirmed using echocardiogram, and the degree of heart failure induction and therapeutic effects were then determined.

### 2. Inhibitory or Alleviating Effects of Compounds on Heart Hypertrophy and Heart Failure

A ratio of heart weight (HW) to body weight (BW) (HW/BW ratio) and a ratio of heart weight (HW) to tibia length (HW/TL ratio) were respectively estimated using echocardiogram.

Further, left ventricular end-diastolic and systolic diameters were measured according to the standard method of the American Society for Echocardiography. Fractional shortening (%) which is an indicator of the ventricular contractility is calculated according to the following equation.

F.S=(left ventricular end-diastolic diameter)−(left ventricular end-systolic diameter)/left ventricular end-diastolic diameter

### Experimental Example 1

### Inhibitory Effects of MB660 on Heart Hypertrophy and Heart Failure

Among compounds of Formula 1, inhibitory effects of 7,8-dihydro-2,2-dimethyl-2H-naphtho(2,3-b)dihydropyran-7,8-dione (hereinafter, referred to as “MB660”) on heart hypertrophy and heart failure were examined. For this purpose, experimental animals were divided into four groups as given in Table 1 below:

SHAM group (banded; control group),

TAC group (banded; experimental group),

Vehicle-treated (−) group, and

Group (+) received the compound of Example 1.

2 days after TAC treatment, animals were given test samples. Induction of heart hypertrophy was carried out over 2 weeks after TAC treatment. Induction of heart failure was carried out over 5 weeks after TAC treatment.

TABLE 2

| Group name | Conditions | Dose | n Number |
|---|---|---|---|
| SHAM MB(−) | Non-banded/MB660 | SLS (10 mg/kg, | 10 |
| | not administered | vehicle) | |
| SHAM MB(+) | Non-banded/MB660 | MB660 (30 mg/kg) | 10 |
| | administered | | |
| TAC MB(−) | Banded/MB660 not | SLS (10 mg/kg, | 10 |
| | administered | vehicle) | |
| TAC MB(+) | Banded/MB660 | MB660 (150 mg/kg) | 10 |
| | administered | | |

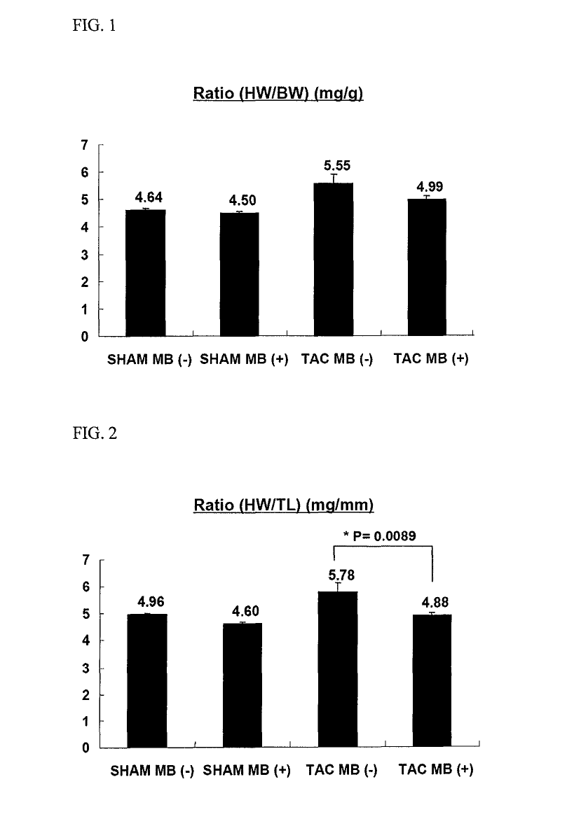

For the heart hypertrophy-induced model, the HW/BW ratio and the HW/TL ratio are shown in FIGS. 1 and 2, respectively, and the fractional shortening is shown in FIG. 3.

Referring to FIGS. 1 and 2, the normal control group (SHAM) exhibited no significant difference with the MB660-treated group and the non-treated group, whereas treatment of MB660 on the group with TAC-induced heart hypertrophy exhibited a significantly low value of heart hypertrophy, as compared to the non-MB660 treated counterpart group, thus being approximate to a level of the normal control group. Referring to FIG. 3, the MB660-treated group exhibited a significant increase of the fractional shortening, as compared to the non-MB660 treated group, thus confirming that the myocardial contractility was improved.

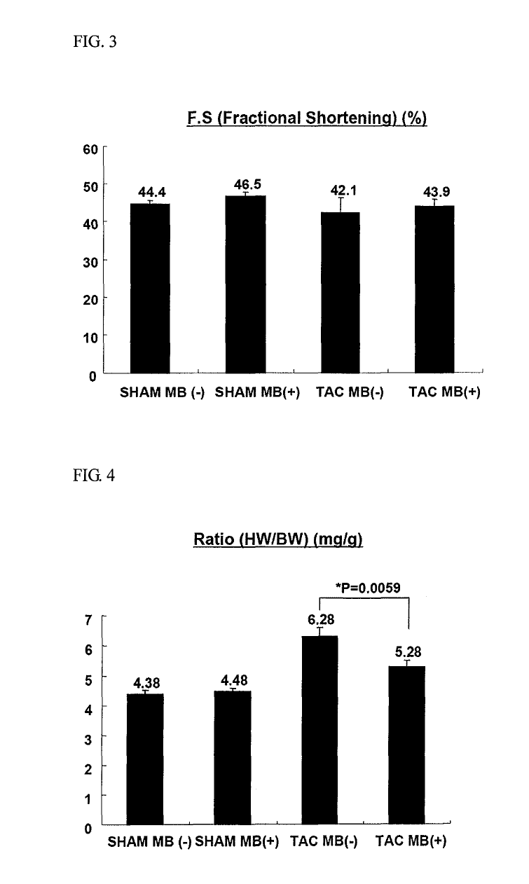

For the heart failure-induced model, the HW/BW ratio and the HW/TL ratio are shown in FIGS. 4 and 5, respectively, and the fractional shortening is shown in FIG. 6.

Referring to FIGS. 4 and 5, the heart failure-induced model with treatment of MB660 also exhibited significantly low values of HW/BW and HW/TL ratios, as compared to the control group (non-MB660 treated group), thus showing characteristics similar to those of the normal control group. From these results, it can be seen that MB660 has inhibitory effects against increases of heart weight due to myocardial hypertrophy or the like. The fractional shortening in FIG. 4 was also significantly increased in the MB660-treated group compared to the non-MB660 treated group.

Taken altogether, administration of the compound in accordance with the present invention results in significant inhibition of heart hypertrophy and heart failure, and therefore the compound in accordance with the present invention can be prophylactically effective for these diseases.

### Experimental Example 2

### Effects of MB660 on Reversal of Heart Hypertrophy and Heart Failure

In order to measure effects of MB660 on the reversal of heart hypertrophy and heart failure, the experiment was carried out as follows. For heart hypertrophy- and heart failure-induced models, experimental animals were divided into two groups as given in Table 2 below:

Vehicle-treated (−) group, and

MB660-administered (+) group.

After induction of heart hypertrophy (2 weeks after TAC treatment) and heart failure (5 weeks after TAC treatment), animals were given test samples for 4 weeks.

TABLE 3

| Group name | Conditions | Dose | n Number |
|---|---|---|---|
| TAC MB(−) | Banded/MB660 not | SLS (10 mg/kg, vehicle) | 10 |
| | administered | | |
| TAC MB(+) | Banded/MB660 | MB660 (150 mg/kg) | 10 |
| | administered | | |

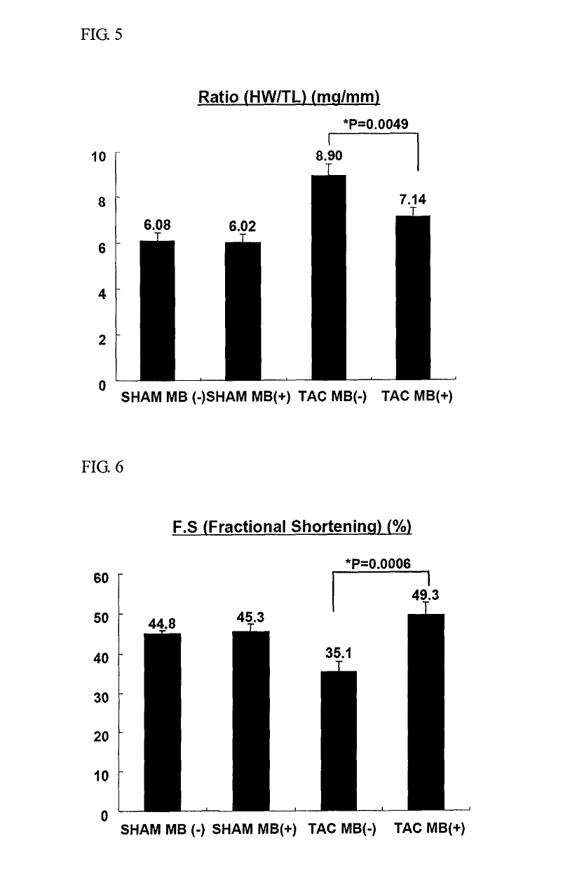

For the heart hypertrophy-induced model, the HW/BW ratio and the HW/TL ratio are shown in FIGS. 7 and 8, respectively, and the fractional shortening is shown in FIG. 9.

Referring to FIGS. 7 and 8, the MB660-treated group exhibited a HW/BW value of 6.23 which is 30% or more lower than the non-MB660 treated group, and a HW/TL value of 7.68 which is about 25% lower than the non-MB660 treated group. Referring to FIG. 9, it can be seen that the MB660-treated group exhibited a significant increase in the fractional shortening, as compared to the non-MB660 treated group.

Therefore, it can be confirmed that the MB660 compound exhibits significant effects on loss of the heart weight and improvement of the myocardial contractility in heart hypertrophy-induced mice, and can therefore effectively used for the treatment of heart hypertrophy.

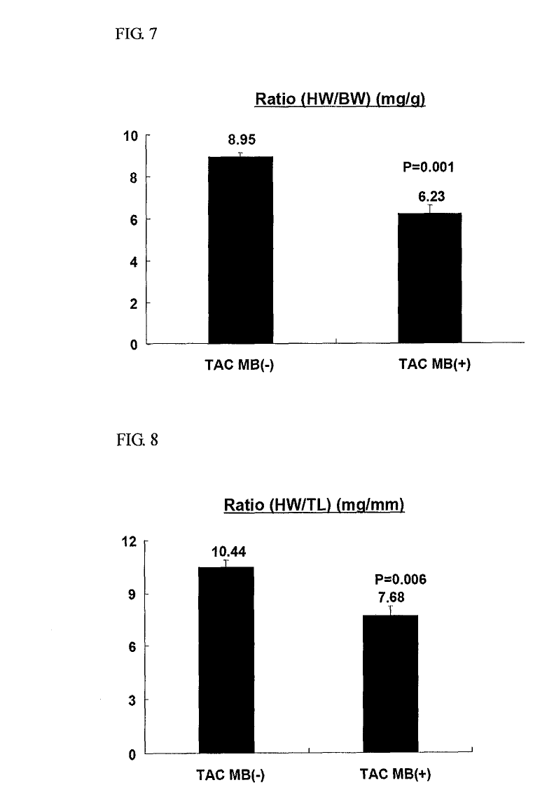

For the heart failure-induced model, the HW/BW ratio and the HW/TL ratio are shown in FIGS. 10 and 11, respectively, and the fractional shortening is shown in FIG. 12.

Referring to FIGS. 10 to 12, it can be confirmed that the MB660-treated group exhibits low values of HW/BW and HW/TL ratios in conjunction with a significant increase of the fractional shortening, as compared to the control group. Therefore, it can be confirmed that the MB660 compound exerts excellent effects on the treatment of heart failure.

### Experimental Example 3

### Changes of Heart Weight in Response to Doses of MB660 in Heart Hypertrophy Models

In order to investigate therapeutic effects of MB660 on heart hypertrophy and heart failure in response to doses of MB660,8-week-old C57BL/6J male mice were subjected to TAC as given in Table 3, and body weight changes, dietary intake and HW/BW ratios were measured with varying doses of MB660 at 30 mg/kg, 60 mg/kg, 100 mg/kg, and 150 mg/kg, respectively. The results obtained are shown in FIGS. 9 and 10. Mice were fed low-fat diet (11.9 kcal % fat, 5053, Labdiet). 2 days after the operation of TAC, animals were orally given test samples for 2 weeks.

TABLE 4

| Group name | Conditions | Dose | n Number |
|---|---|---|---|
| TAC | Banded/MB660 not | Sterile water | 10 |
| | administered | | |
| 30 | Banded/MB660 | MB660 (30 mg/kg) | 10 |
| | administered | | |
| 60 | Banded/MB660 | MB660 (60 mg/kg) | 10 |
| | administered | | |
| 100 | Banded/MB660 | MB660 (100 mg/kg) | 10 |
| | administered | | |
| 150 | Banded/MB660 | MB660 (150 mg/kg) | 10 |
| | administered | | |

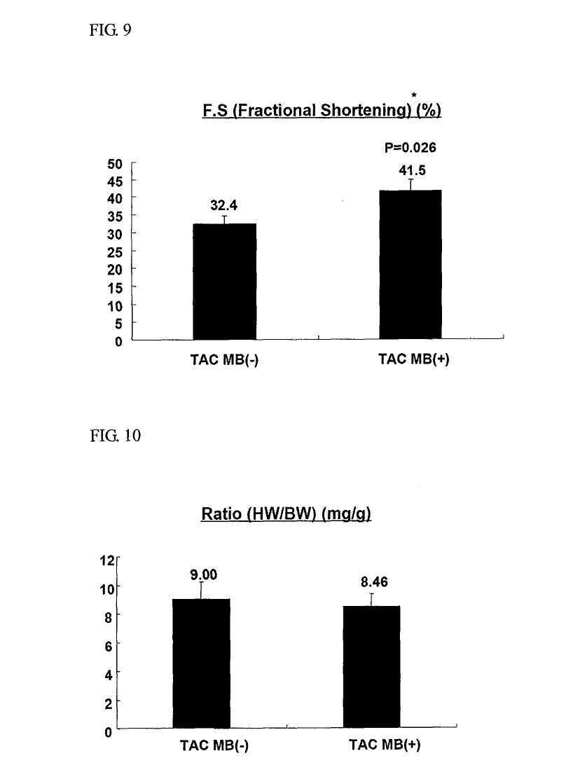

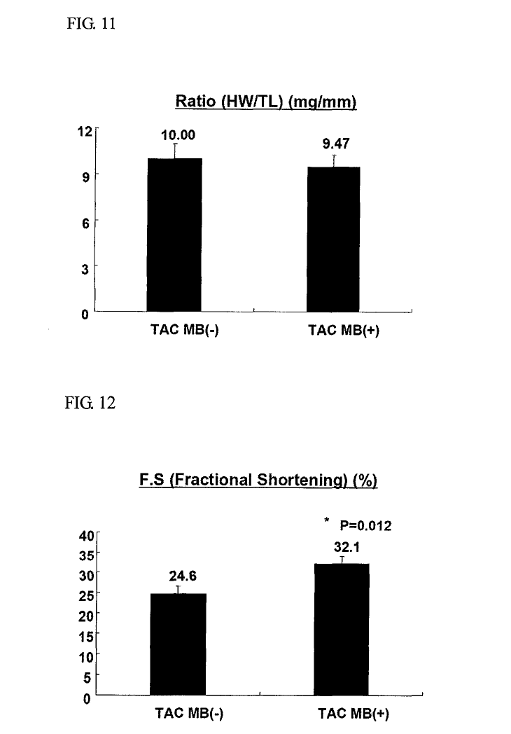

Although all the groups exhibited no significant difference in body weight and dietary intake (see FIG. 13), the MB660-administered group exhibited a significant decrease of a HW/BW ratio as compared to the control group (TAC) (see FIG. 14), thus confirming that such a decrease of the HW/BW ratio was due to loss of the heart weight. Even though the HW/BW ratio was increased at 60 mg of MB660 as compared to the group with administration of 30 mg of MB660, the HW/BW ratio was generally decreased with an increasing dose of MB660.

### Experimental Example 4

### Changes of Heart Size in Heart Hypertrophy Models

In order to examine changes of heart size in response to the administration of MB660, experimental animals were divided into four groups as given in Table 4 below:

SHAM group (non-banded; control group),

TAC group (banded; experimental group),

Vehicle-treated (−) group, and

Group (+) received the compound of Example 1.

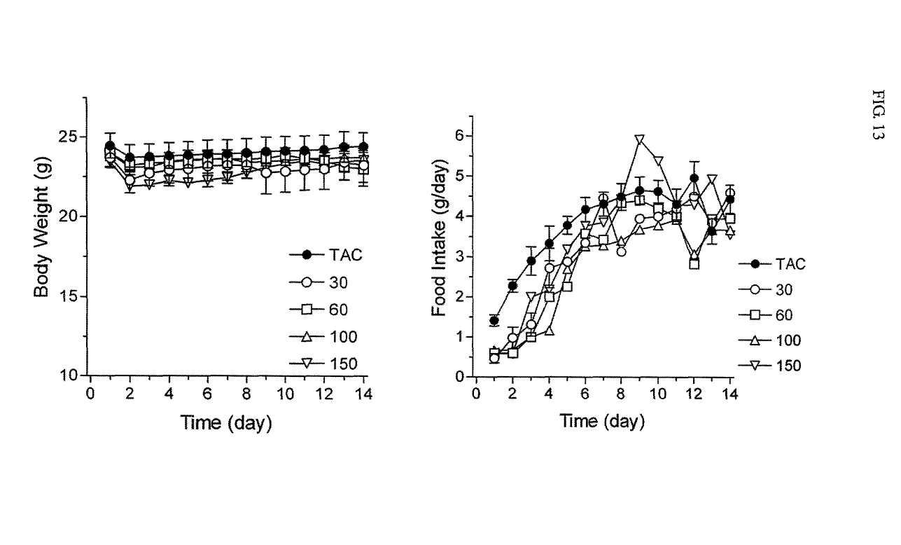

One week after TAC treatment, animals were orally given MB660 for two weeks. Three weeks after TAC treatment, animals were sacrificed and the heart was excised, followed by size examination. The results obtained are shown in FIG. 15.

TABLE 5

| Group name | Conditions | Dose | n Number |
|---|---|---|---|
| SHAM MB(−) | Non-banded/MB660 | SLS (10 mg/kg, | 10 |
| | not administered | vehicle) | |
| SHAM MB(+) | Non-banded/MB660 | MB660 (30 mg/kg) | 10 |
| | administered | | |
| TAC MB(−) | Banded/MB660 not | SLS (10 mg/kg, | 10 |
| | administered | vehicle) | |
| TAC MB(+) | Banded/MB660 | MB660 (150 mg/kg) | 10 |
| | administered | | |

Referring to FIG. 15, the normal control group exhibited no significant difference in response to the administration of MB660, whereas the control group (non-administration of MB660 after TAC treatment) exhibited a very significant increase of the heart size due to myocardial hypertrophy, and the MB660-administered group exhibited a significant decrease of the heart size, similar to that of the normal control group (SHAM group).

### Experimental Example 5

### Mitochondrial Changes in Heart Hypertrophy and Heart Failure Models

Animals were orally given test samples for 4 weeks after induction of heart hypertrophy (2 weeks after TAC treatment) and heart failure (5 weeks after TAC treatment). The left ventricular myocardial tissues of animals were fixed in 2.5% glutaraldehyde to prepare tissue sections, and mitochondrial morphological changes were examined under a transmission electron microscope.

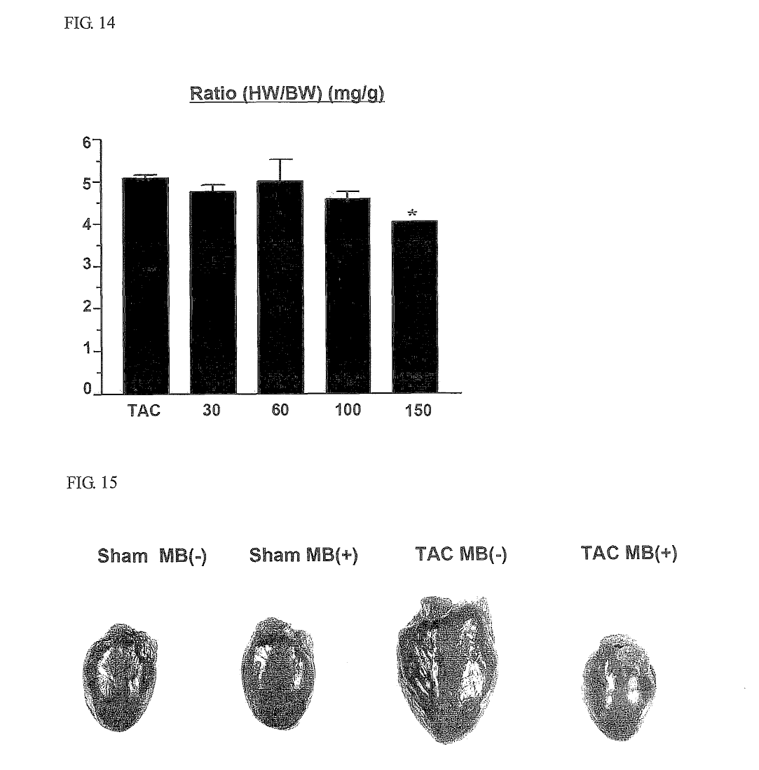

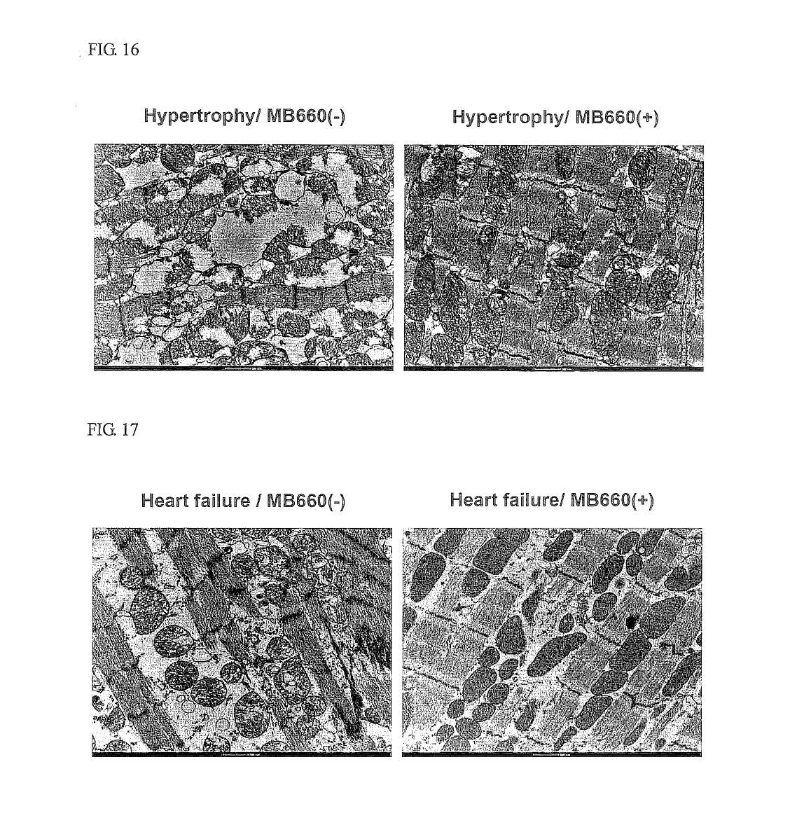

In C57BL/6J mice with TAC-induced heart hypertrophy and heart failure, alterations in cardiomyocytic mitochondria in response to the administration of MB660 were observed. The results obtained are shown in FIGS. 16 and 17.

Referring to FIGS. 16 and 17, mitochondrial abnormalities were observed due to cardiomyocytic apoptosis and deficiency of available oxygen (see left panels) upon the occurrence of heart hypertrophy and heart failure, but mitochondrial function returned to the original condition by administration of MB660 (see right panels).

### INDUSTRIAL APPLICABILITY

As apparent from the foregoing, a pharmaceutical composition in accordance with the present invention inhibits the occurrence of myocardial hypertrophy to thereby exhibit significant effects on the reduction of heart weight and size. Therefore, the pharmaceutical composition of the present invention has excellent effects on the treatment and prevention of cardiac diseases such as heart hypertrophy, heart failure, etc.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A pharmaceutical composition for the treatment and prevention of cardiac diseases, comprising:
(a) a therapeutically effective amount of one or more selected from compounds represented by Formulae 1 and 2: or a pharmaceutically acceptable salt, prodrug, solvate or isomer thereof; and (b) a pharmaceutically acceptable carrier, diluent or excipient or any combination thereof. 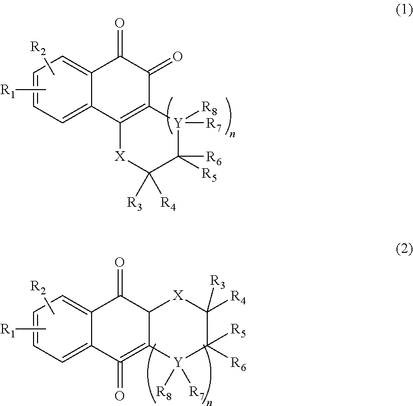
wherein:
R₁ and R₂ are each independently hydrogen, halogen, hydroxyl, or C₁-C₆ lower alkyl or alkoxy, or R₁ and R₂ may be taken together to form a substituted or unsubstituted cyclic structure which may be saturated or partially or completely unsaturated;
R₃, R₄, R₅, R₆, R₇ and R₈ are each independently hydrogen, hydroxyl, C₁-C₂₀ alkyl, alkene or alkoxy, or C₄-C₂₀ cycloalkyl, heterocycloalkyl, aryl or heteroaryl, or two of R₃ to R₈ may be taken together to form a cyclic structure which may be saturated or partially or completely unsaturated;
X is selected from the group consisting of C(R)(R′), N(R″) wherein R, R′ and R″ are each independently hydrogen or C₁-C₆ lower alkyl, O and S;
Y is C, S or N, with the proviso that R₇ and R₈ are absent when Y is S, and R₇ is hydrogen or C₁-C₆ lower alkyl and R₈ is absent when Y is N; and
n is 0 or 1, with the proviso that when n is 0, carbon atoms adjacent to n form a cyclic structure via a direct bond.

2. The composition according to claim 1, wherein X is O.

3. The composition according to claim 1, wherein the prodrug is a compound represented by Formula 1a below: 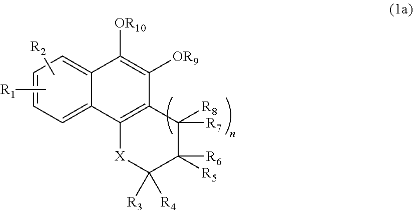
wherein,
R₁, R₂, R₃, R₄, R₅, R₆, R₇, R₈, X and n are as defined in Formula 1;
R₉ and R₁₀ are each independently —SO₃—Na⁺ or substituent represented by Formula A below or a salt thereof, 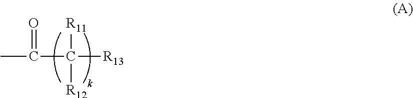
wherein,
R₁₁ and R₁₂ are each independently hydrogen or substituted or unsubstituted C₁-C₂₀ linear alkyl or C₁-C₂₀ branched alkyl,
R₁₃ is selected from the group consisting of substituents i) to viii) below,
i) hydrogen;
ii) substituted or unsubstituted C₁-C₂₀ linear alkyl or C₁-C₂₀ branched alkyl;
iii) substituted or unsubstituted amine;
iv) substituted or unsubstituted C₃-C₁₀ cycloalkyl or C₃-C₁₀ heterocycloalkyl;
v) substituted or unsubstituted C₄-C₁₀ aryl or C₄-C₁₀ heteroaryl;
vi) —(CRR′—NR″CO)₁—R₁₄, wherein R, R′ and R″ are each independently hydrogen or substituted or unsubstituted C₁-C₂₀ linear alkyl or C₁-C₂₀ branched alkyl, R₁₄ is selected from the group consisting of hydrogen, substituted or unsubstituted amine, cycloalkyl, heterocycloalkyl, aryl and heteroaryl, 1 is selected from the 1˜5;
vii) substituted or unsubstituted carboxyl;
viii) —OSO₃-Na⁺;
k is selected from the 0˜20, with proviso that when k is 0, R₁₁ and R₁₂ are not anything, and R₁₃ is directly bond to a carbonyl group.

4. The composition according to claim 1, wherein the compound of Formula 1 is selected from compounds of Formulas 3 and 4 below: 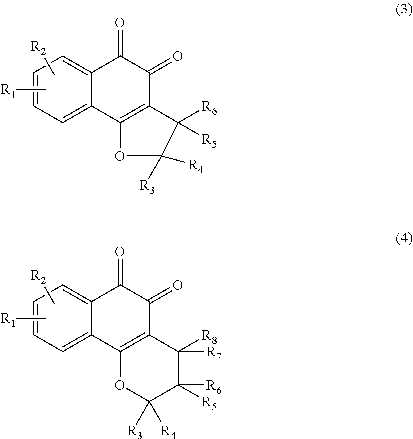
wherein R₁, R₂, R₃, R₄, R₅, R₆, R₇ and R₈ are as defined in Formula 1.

5. The composition according to claim 1, wherein each of R₁ and R₂ is respectively hydrogen.

6. The composition according to claim 4, wherein the compound of Formula 3 is a compound of Formula 3a below in which R₁, R₂ and R₄ are respectively hydrogen, or a compound of Formula 3b below in which R₁, R₂ and R₆ are respectively hydrogen:

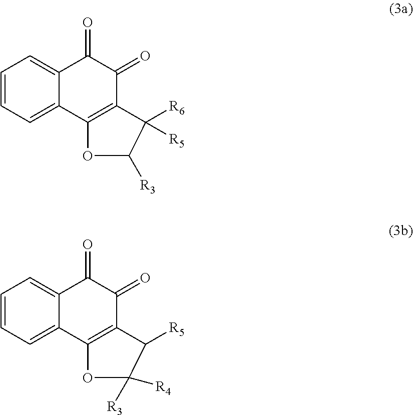

7. The composition according to claim 4, wherein the compound of Formula 4 is a compound of Formula 4a below in which R₁, R₂, R₅, R₆, R₇ and R₈ are respectively hydrogen:

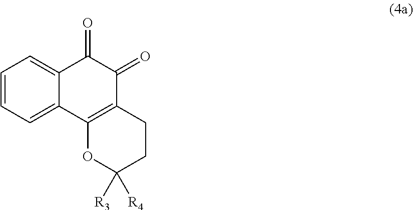

8. The composition according to claim 1, wherein the compound of Formula 2 is a compound of Formula 2a in which n is 0 and adjacent carbon atoms form a cyclic structure via a direct bond therebetween and Y is C, or a compound of Formula 2b in which n is 1 Y is C: 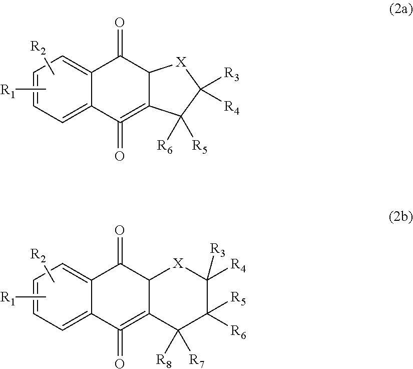
wherein R₁, R₂, R₃, R₄, R₅, R₆, R₇, R₈ and X are as defined in Formula 1.

9. The composition according to claim 1, wherein the compound of Formula 1 or Formula 2 is contained in a crystalline structure.

10. The composition according to claim 1, wherein the compound of Formula 1 is contained in an amorphous structure.

11. The composition according to claim 1, wherein the compound of Formula 1 or Formula 2 is formulated into the form of a fine particle.

12. The composition according to claim 11, wherein the formulation for form of a fine particle is carried out by using the particle micronization method selected from the group consisting of mechanical milling, spray drying, precipitation method, homogenization, and supercritical micronization.

13. The composition according to claim 12, wherein the formulation is carried out by using jet milling as a mechanical milling and/or spray drying.

14. The composition according to claim 11, wherein the particle size of fine particles is 5 nm to 500 μm.

15. The composition according to claim 1, wherein the pharmaceutical composition is prepared into an intestine-targeted formulation.

16. The composition according to claim 15, wherein the intestine-targeted formulation is carried out by addition of a pH sensitive polymer.

17. The composition according to claim 15, wherein the intestine-targeted formulation is carried out by addition of a biodegradable polymer which is decomposable by an intestine-specific bacterial enzyme.

18. The composition according to claim 15, wherein the intestine-targeted formulation is carried out by addition of a biodegradable matrix which is decomposable by an intestine-specific bacterial enzyme.

19. The composition according to claim 15, wherein the intestine-targeted formulation is carried out by a configuration with time-course release of the drug after a lag time (‘time-specific delayed-release formulation’).

20. The composition according to claim 1, wherein the cardiac disease is selected from the group consisting of heart hypertrophy, heart failure, congestive heart failure, angina pectoris, and myocardial infarction.

21. A method for preparing a medicine for the treatment and/or prevention of cardiac diseases using the compound of Formula 1 according to claim 1.

22. The composition according to claim 1, wherein cardiac diseases is the compound of cardiac hypertrophy or heart failure.
